(19) United States
(12) Patent Application Publication
(10) Pub. No.: US 2013/0078255 A1
(43) Pub. Date: Mar. 28, 2013

(54) SERUM AND TISSUE BIOMARKERS OF HUMAN HCC

(71) Applicant: Jürgen Borlak, Lehrte/Immensen (DE)

(72) Inventor: Jürgen Borlak, Lehrte/Immensen (DE)

(21) Appl. No.: 13/637,751

(22) PCT Filed: Mar. 29, 2011
(86) PCT No.: PCT/EP11/01683

§ 371 Date: Dec. 7, 2012

(30) Foreign Application Priority Data
Mar. 29, 2010 (EP) 10 075 140.3

(51) Int. Cl. G01N 33/574; C07K 14/75; C07K 14/435

(52) U.S. Cl. CPC G01N 33/574; C12Y 301/01; C12Y 111/01; C12Y 301/08001; C07K 14/435; C07K 14/75
USPC 424/139.1; 435/198; 530/400; 530/359; 435/192; 530/387.9; 536/24.5; 536/23.1; 536/23.2; 435/7.1; 436/501; 435/6.12; 435/196; 530/350; 530/381

(57) ABSTRACT

The application is based on the surprising finding that proteins regulated by increased c-myc activity in the liver can be used as sis and/or treatment monitoring of cancer and dysplasia, in particular of liver cell dysplasia and hepatocellular carcinoma (HCC), and wherein the proteins are selected from a first group consisting of Polymeric immunoglobulin receptor, Phosphatidylinositol—glycan—specific phospholipase D, Alpha—fetoprotein, Antithrombin 3, Apolipoprotein E, Apolipoprotein M, Fibrinogen beta-chain, Haptoglobin, Paraoxonase 1, Retinol binding protein, Serum amyloid P- component, Transthyretin, or from a second group consisting of Afamin, Glutathione peroxidase 3, Hemopexin, Major urinary protein, Serine protease inhibitor A3K. Consequently, medical uses of said proteins, of corresponding compositions, of corresponding antibodies, of corresponding siRNA and of corresponding nucleotide sequences are claimed. Also claimed are corresponding kits and corresponding methods and procedures.

The invention is directed to novel biomarkers of dysplasia and cancer and the use thereof, in particular in the diagnosis, prognosis and/or treatment monitoring of liver cell dysplasia or hepatocellular carcinoma (HCC). Areas of application are the life sciences: biology, biochemistry, biotechnology, medicine and medical technology.

Liver cancer is the fifth most common cancer worldwide and the third most common cause of cancer mortality [1]. At least 500,000 people are diagnosed with HCC each year with an extraordinary high incidence in sub-Saharan Africa and Southeast Asia. According to the American Cancer Society, 21,370 new cases (15,190 in men and 6,180 in women) of primary liver and intrahepatic bile duct cancer were diagnosed in the United States in 2008 alone with an expected 5-year survival of less than 8 percent [2]. So far, the development of effective strategies for cancer diagnosis and treatment has lagged behind. Nonetheless, diagnosis at early stages of disease would improve overall survival, but imaging and other non-invasive methods are still not sufficiently sensitive. Furthermore, cancer patients present symptoms only at advanced stages of disease.

Ideally, sensitive biomarkers allow early detection of disease [3] but only a few serum markers of HCC are used routinely, this may include alpha-fetoprotein (AFP), lens culinaris agglutinin-reactive AFP (AFP-L3), des-gamma carboxyprothrombin (DCP), prothrombin induced by vitamin K absence-II (PIVKA-II), carcinoembryonic antigen (CEA), as well as the carbohydrate antigen (CA) 125 and 15-3 [4,5,6]. Unfortunately, their regulation in several other malignancies and the high variability amongst individual patients renders them less specific [7,8,9]. Indeed, over the past 15 years very few serum tumor markers have been introduced to the clinic and a recent study suggested AFP to be more sensitive than DCP and AFP-L3% for the diagnosis of early stage HCC [6].

Specifically, Myc over-expression has been observed in the pathogenesis of many cancers including liver while impairment of Myc hyperactivity was sufficient to stop tumor growth [10]. The search for regulated serum proteins in a genetic model of liver cancer may facilitate an identification of novel biomarkers for translational research.

The aim of the present invention is therefore to provide body fluid and tissue biomarkers and molecules binding to said biomarkers, compositions and a kit, as well as methods for the diagnosis, prognosis and/or monitoring the treatment of dysplasia and cancer, in particular of liver cell dysplasia and hepatocellular carcinoma (HCC).

To this end, the implementation of the embodiments and actions as described in the claims provides appropriate means to fulfill these demands in a satisfying manner.

The invention is based on the surprising finding that proteins regulated by increased c-myc activity in the liver may be used as body fluid markers and/or as tissue biomarkers in the diagnosis, prognosis and/or treatment monitoring of cancer and dysplasia, in particular of liver cell dysplasia and hepatocellular carcinoma (HCC), and wherein the proteins are selected from the group consisting of Polymeric immunoglobulin receptor, Phosphatidylinositol-glycan-specific phospholipase D, Afamin, Alpha-fetoprotein, Antithrombin 3, Apolipoprotein E, Apolipoprotein M, Fibrinogen beta-chain, Haptoglobin, Paraoxonase 1, Retinol binding protein, Serum amyloid P-component, Transthyretin, Glutathione peroxidase 3, Hemopexin, Major urinary protein, Serine protease inhibitor A3K, wherein the proteins Polymeric immunoglobulin receptor, Phosphatidylinositol-glycan-specific phospholipase D and Afamin or a combination thereof is/are particularly preferred, in particular if said protein(s) is/are combined with at least one other protein of said 17 proteins.

In a first aspect the invention is thus directed to a protein regulated by increased c-myc activity in the liver for use as biomarker, in particular as body fluid marker and/or as tissue marker, in the diagnosis, prognosis and/or monitoring the treatment of liver cell dysplasia or hepatocellular carcinoma (HCC), preferably in the premalignant or in the early stage of HCC, wherein the protein is selected from a first group consisting of Polymeric immunoglobulin receptor, Phosphatidylinositol-glycan-specific phospholipase D, Alpha-fetoprotein, Antithrombin 3, Apolipoprotein E, Apolipoprotein M, Fibrinogen beta-chain, Haptoglobin, Paraoxonase 1, Retinol binding protein, Serum amyloid P-component, Transthyretin, or from a second group consisting of Afamin, Glutathione peroxidase 3, Hemopexin, Major urinary protein, Serine protease inhibitor A3K.

The proteins according to the invention concern gene products of mammalia, preferably gene products of the genome of mus musculus or homo sapiens, in particular the respective gene products of homo sapiens are preferred, or, respectively, sequence fragments of said gene products as described herein.

According to the invention the term “regulated by increased c-myc acitivity in the liver” as common technical feature of the 17 biomarker proteins mentioned herein, said term is particularly directed to the regulation of said proteins by c-myc overexpression in tissue of the liver. Within the context of the methods mentioned hereinafter, the term “increased c-myc activity” is directed to any alteration of c-myc which causes cancer, such as overexpression or mutations of c-myc or excessive activation of c-myc triggered by growth factors or mitogenic signals (e.g. EGF, Wnt or Shh), and molecules of their signalling pathways (e.g. MAPK/ERK pathway).

The term “dysplasia” according to the invention is directed to low grade and/or high grade dysplasia, wherein “low grade dysplasia” is particularly directed to a lesion having minimal aberration inside the cell, and “high grade dysplasia” also comprises mild or medium dysplasia. The term “liver cell dysplasia” according to the invention is in particular directed to premalignant lesions of HCC, as described for example by Kojiro M. J Hepatobiliary Pancreat Surg. 2000;7(6):535-41.

Another aspect of the invention is directed to a protein regulated by increased c-myc activity in the liver for the use as biomarker, in particular as body fluid marker and/or as tissue marker, in the diagnosis, prognosis and/or treatment monitoring of dysplasia or cancer, in particular breast, colon, lung and stomach dysplasia or cancer, or preferably leukaemia, glioblastoma or neuroblastoma, wherein the protein is selected from a first group consisting of Polymeric immunoglobulin receptor, Phosphatidylinositol-glycan-specific phospholipase D, or from a second group consisting of Afamin, and, if Afamin is selected as protein, Afamin is used as body fluid marker.

Within the context of the invention, Polymeric immunoglobulin receptor may be used as a surface marker of dysplastic or tumor cells and/or Phosphatidylinositol-glycan-specific phospholipase D may be used as a marker enzyme for dysplastic or tumor cells, wherein as enzyme substrate GPI-anchored proteins may be used.

The term “body fluid” according to the invention is directed to any body fluid of a subject, in particular to blood, plasma, serum or urine, whereas blood serum or plasma is the preferred body fluid within the context of the invention.

The term “tissue” according to the invention is directed to the cellular organizational level intermediate between cells and the complete organism, in particular to an ensemble of cells from the same origin that together carry out a specific biological function, and wherein the tissue may be either part of an animal or human body or wherein preferably the tissue has been removed from an animal or human body.

Accordingly, the proteins according to the invention are preferably used as immunohistochemical markers, such as for a immunohistochemical staining, or as blood plasma markers or particularly as blood serum markers.

The invention is further directed to molecules specifically binding to the protein biomarkers mentioned herein or to mRNA coding for said proteins, and wherein said molecules are selected from the group consisting of antibodies and siRNA. The terms “specifically binding” or “specific for” as mentioned herein is particularly related to an association of the biomarker or mRNA with the binding molecule being established via an association constant Ka>1000 M⁻¹.

Thus, the invention is also directed to an antibody specific for a protein regulated by increased c-myc activity in the liver, wherein the protein is selected from the group consisting of Polymeric immunoglobulin receptor, Phosphatidylinositol-glycan-specific phospholipase D, Afamin, Alpha-fetoprotein, Antithrombin 3, Apolipoprotein E, Apolipoprotein M, Fibrinogen beta-chain, Haptoglobin, Paraoxonase 1, Retinol binding protein, Serum amyloid P-component, Transthyretin, Glutathione peroxidase 3, Hemopexin, Major urinary protein, Serine protease inhibitor A3K, and wherein the antibody is for use in the diagnosis, prognosis and/or treatment monitoring of liver cell dysplasia or HCC or, if the protein is selected from the group consisting of Polymeric immunoglobulin receptor, Phosphatidylinositol-glycan-specific phospholipase D, and Afamin, for use in the diagnosis, prognosis and/or treatment monitoring of dysplasia or cancer, in particular breast, colon, lung and stomach dysplasia or cancer, or preferably leukemia, glioblastoma or neuroblastoma, and wherein, if Afamin is selected, the anti Afamin antibody is used for serum profiling

In particular, according to the invention, it is preferred if said antibodies are used for serum profiling in the diagnosis, prognosis and/or treatment monitoring of liver cell dysplasia or HCC or, if the antibody is specfic for a protein selected from the group consisting of of Polymeric immunoglobulin receptor, Phosphatidylinositol-glycan-specific phospholipase D, Afamin, for serum profiling in the diagnosis, prognosis and/or treatment monitoring of dysplasia or cancer, in particular breast, colon, lung and stomach dysplasia or cancer, or preferably leukemia, glioblastoma or neuroblastoma.

Within the inventive context, antibodies are understood to include monoclonal antibodies and polyclonal antibodies and antibody fragments (e.g., Fab, and F(ab′)₂) specific for one of said proteins. Polyclonal antibodies against selected antigens may be readily generated by one of ordinary skill in the art from a variety of warm-blooded animals such as horses, cows, goats, rabbits, mice, rats, chicken or preferably of eggs derived from immunized chicken. Monoclonal antibodies may be generated using conventional techniques (see Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988, which are incorporated herein by reference).

The term “serum profiling” according to the invention is in particular directed to the analysis of blood plasma or blood serum for the presence or concentration of the selected protein in said plasma or serum, such as by using a biosensor, performing an ELISA, a Western Blot, a magnetic bead separation/purification, a ZipTip approach, and wherein said procedures, if applicable, may be combined with a mass spectrometry analysis.

The invention is also directed to siRNA, which reduces or preferably inhibits the expression of a protein regulated by increased c-myc activity in the liver, for use in the treatment of liver cell dysplasia or HCC, wherein the protein is selected from the group of consisting of Polymeric immunoglobulin receptor, Phosphatidylinositol-glycan-specific phospholipase D, Alpha-fetoprotein, Antithrombin 3, Apolipoprotein E, Apolipoprotein M, Fibrinogen beta-chain, Haptoglobin, Paraoxonase 1, Retinol binding protein, Serum amyloid P-component, and Transthyretin.

Within this context, the present invention employs siRNA for use in modulating the level of protein presence in the cell, wherein siRNA oligonucleotides specifically hybridize nucleic acids encoding the selected protein and interfere with the expression of the gene coding for said protein.

Preferably, the siRNA comprises double stranded RNA including a sense RNA strand and an antisense RNA strand, wherein the sense RNA strand comprises a subsequence being 19, 20, 21, 22, 23, 24, or 25 contiguous RNA nucleotides in length, preferably corresponding to the ORF sequence encoding the selected protein, and wherein said subsequence contains sequences that are complementary and non-complementary to at least a portion of the mRNA coding for the selected protein.

In another aspect, the invention is directed to a nucleotide sequence coding for a protein regulated by increased c-myc activity in the liver for use in the treatment of liver cell dysplasia or HCC, wherein the protein is selected from the group of Afamin, Glutathione peroxidase 3, Hemopexin, Major urinary protein, and Serine protease inhibitor A3K

The nucleotide sequence particularly comprises a nucleic acid being from about 20 base pairs to about 100,000 base pairs in length. Preferably the nucleic acid is from about 50 base pairs to about 50,000 base pairs in length. More preferably the nucleic acid is from about 50 base pairs to about 10,000 base pairs in length. Most preferred is a nucleic acid from about 50 pairs to about 4,000 base pairs in length. The nucleotide sequence can be a gene or gene fragment that encodes the protein, an oligopeptide or a peptide. Preferably, the nucleotide sequence of the present invention may comprise a DNA construct capable of generating the selected protein and may further include an active constitutive or inducible promoter sequence.

In particular the nucleotide composition comprises a nucleotide sequence encoding a polypeptide which has at least 70% identity, preferably at least 80% identity, more preferably at least 90% identity, yet more preferably at least 95% identity, to the amino acid sequence of the selected protein. In this regard, nucleotide sequences coding for polypeptides which have at least 97% identity are highly preferred, whilst those with at least 98-99% identity are more preferred, and those with at least 99% identity are most preferred. In particular, it is preferred if the nucleotide sequence encodes a polypeptide with 100% identity to the entire amino acid sequence of the selected protein.

In particular, the nucleotide composition comprises a DNA sequence that has at least 70% identity, preferably at least 80% identity, more preferably at least 90% identity, yet more preferably at least 95% identity, to the ORF (or coding sequence, respectively) of one of HNF6 over the entire coding region. In this regard, nucleoetide sequences which have at least 97% identity are highly preferred, whilst those with at least 98-99% identity are more highly preferred, and those with at least 99% identity are most highly preferred. In particular, it is preferred if the nucleotide sequence encodes a DNA sequence that has 100% identity to the entire ORF of HNF6 over the entire coding region.

In another aspect the nucleotide sequence composition may further comprise an enhancer element and/or a promoter located 5′ to and controlling the expression of said therapeutic nucleotide sequence or gene. The promoter is a DNA segment that contains a DNA sequence that controls the expression of a gene located 3′ or downstream of the promoter. The promoter is the DNA sequence to which RNA polymerase specifically binds and initiates RNA synthesis (transcription) of that gene, typically located 3′ of the promoter.

Further, within the context of the present invention an antisense composition is provided, wherein the antisense composition comprises a nucleotide sequence complementary to a coding sequence of Foxa2.

Said nucleotide sequences and siRNA according to the invention may be prepared by any standard method for producing a nucleotide sequence or siRNA, such as by recombinant methods, in particular synthetic nucleotide sequences and siRNA is preferred.

Said nucleotide sequences and siRNA are preferably for the use in the treatment of liver cell dysplasia or HCC by administering an amount of said nucleotide sequences and SiRNA to a subject suffering from or being susceptible to liver cell dysplasia or HCC for decreasing or increasing the expression or biological activity of the targeted protein to a normal level.

The invention thus also relates to a composition comprising a substance that
decreases or inhibits the expression or biological activity of a protein selected from the group consiting of Polymeric immunoglobulin receptor, Phosphatidylinositol-glycan-specific phospholipase D, Alpha-fetoprotein, Antithrombin 3, Apolipoprotein E, Apolipoprotein M, Fibrinogen beta-chain, Haptoglobin, Paraoxonase 1, Retinol binding protein, Serum amyloid P-component, and Transthyretin, and/or
increases the expression or biological activity of a protein selected from the group consiting of Afamin, Glutathione peroxidase 3, Hemopexin, Major urinary protein, Serine protease inhibitor A3K, for the treatment of liver cell dysplasia and HCC, wherein the substance is preferably selected from the group consisting of said nucleotide sequences and siRNA according to the invention.

Preferably, the compositions according to the invention further comprise a pharmaceutically acceptable carrier and/or recipient and/or diluent.

The term “biological activity” within the context of the invention is particularly directed to the interaction of the selected protein with other biomolecules, in particular with proteins, carbohydrates and lipids or with a combination thereof.

The term “subject”, as used herein, is directed to a mammal, in particular to a mouse or a human being having or being susceptible to dysplasia or cancer, preferably liver cell dysplaisa or HCC, more particular to a human dysplasia or cancer patient or a transgenic cancer mouse, such as a patient having liver cell dysplasia or HCC or a c-myc- transgenic mouse may be.

The invention is also directed to a method of detecting liver cell dysplasia or HCC, or of predicting the susceptibility or resistance to liver cell dysplasia or HCC, comprising testing a sample isolated from the liver or from body fluid, preferably a tissue sample or a blood serum or plasma sample, of a subject for the presence or concentration of a protein selected from a first group consisting of Polymeric immunoglobulin receptor, Phosphatidylinositol-glycan-specific phospholipase D, Alpha-fetoprotein, Antithrombin 3, Apolipoprotein E, Apolipoprotein M, Fibrinogen beta-chain, Haptoglobin, Paraoxonase 1, Retinol binding protein, Serum amyloid P-component, and Transthyretin, or from a second group consisting of Afamin, Glutathione peroxidase 3, Hemopexin, Major urinary protein, and Serine protease inhibitor A3K, and wherein, in particular, the sample is tested for the increase of a protein selected from said first group and/or the decrease of a protein selected from the said second group.

The invention is further directed to a method of detecting the response to a compound in the treatment of liver cell dysplasia or HCC, or of predicting the responsiveness to a compound in the treatment of liver cell dysplasia or HCC, comprising determining the presence or concentration of a protein regulated by increased c-myc activity in the liver, wherein the protein is selected from a first group consisting of Polymeric immunoglobulin receptor, Phosphatidylinositol-glycan-specific phospholipase D, Alpha-fetoprotein, Antithrombin 3, Apolipoprotein E, Apolipoprotein M, Fibrinogen beta-chain, Haptoglobin, Paraoxonase 1, Retinol binding protein, Serum amyloid P-component, and Transthyretin, or from a second group consisting of Afamin, Glutathione peroxidase 3, Hemopexin, Major urinary protein, and Serine protease inhibitor A3K, in a sample isolated from the liver or from body fluid, preferably a tissue sample or a blood serum or plasma sample, of a subject treated with a compound, wherein the compound is selected from the group consisting of c-myc activity modulator, siRNA, and nucleotide sequence as mentioned herein, and if applicable in combination with a chemotherapeutic drug, and wherein the sample is preferably tested for the decrease of the presence or concentration of a protein selected from said first group and/or the increase of the presence or concentration of a protein selected from said second group.

The term “c-myc activity modulator” or “compound for modulating c-myc activity” or “compound to be tested” according to the invention is in particular directed to antisense oligonucleotides inhibiting c-myc expression, e.g. c-myc antisense phosphorothioate oligonucleotides, and to inhibitors of c-myc/max dimerization, e.g. small organic molecules such as [Z,E]-5[4-ethylbenzylidine]-2-thioxothiazolidin-4-one or pyrazolo[1,5-a]pyrimidines, and to inhibitors of growth factors or mitogenic signals (e.g. EGF, Wnt or Shh), and their cell receptors (e.g. EGFR) triggering signals resulting in the activation of c-myc, e.g. Sorafenib, Sunitinib, Gefitinib, Erlotinib, anti-HER1 antibody, anti-HER2 antibody, anti-HER3 antibody, anti-HER4 antibody, Trastuzumab (Herceptin), Cetuximab, Panitumumab, Matuzumab, Nimotuzumab, MDX-447, Pertuzumab.

According to the invention, as chemotherapeutic drugs in particular any antineoplastic chemotherapy drugs usable for treating HCC and any chemopreventive drugs usable for treating liver cell dysplasia are suitable, and wherein the antineoplastic chemotherapy drug is preferably selected from the group consisiting of Taxol, 5-fluorouracil, doxorubicin and vinblastine, or wherein the chemopreventive drug is preferably selected from the group consisiting of Zileuton and Celecoxib.

The invention further concerns a method to screen for and to identify a compound for modulating c-myc activity in the liver of a subject suffering from or being susceptible to liver cell dysplasia or HCC, comprising the use of a protein biomarker selected from the group consisting of Polymeric immunoglobulin receptor, Phosphatidylinositol-glycan-specific phospholipase, D, Afamin, Alpha-fetoprotein, Antithrombin 3, Apolipoprotein E, Apolipoprotein M, Fibrinogen beta-chain, Haptoglobin, Paraoxonase 1, Retinal binding protein, Serum amyloid P-component, Transthyretin, Glutathione peroxidase 3, Hemopexin, Major urinary protein, Serine protease inhibitor A3K and/or the use of an antibody specific for one of said proteins. Thus, the invention is also directed to the use of at least one of said proteins and/or of at least one of said antibodies to screen for and to identify a compound for modulating c-myc activity in the liver of a subject suffering from or being susceptible to liver cell dysplasia or HCC, in particular by a body body fluid analysis of the subject to which a compound, in particular a (at least putative) c-myc activity modulator, to be tested has been administered.

In another aspect, the invention is directed to a method of qualifying the c-myc activity in a subject, comprising determining in a sample of the liver or in a body fluid sample, preferably in a tissue sample or in a blood serum or plasma sample, of a subject suffering from or being susceptible to liver cell dysplasia or HCC at least one protein selected from a first group consisting of Polymeric immunoglobulin receptor, Phosphatidylinositol-glycan-specific phospholipase D, Alpha-fetoprotein, Antithrombin 3, Apolipoprotein E, Apolipoprotein M, Fibrinogen beta-chain, Haptoglobin, Paraoxonase 1, Retinol binding protein, Serum amyloid P-component, and Transthyretin, and/or at least one protein selected from a second group consisting of Afamin, Glutathione peroxidase 3, Hemopexin, Major urinary protein, and Serine protease inhibitor A3K, wherein the level of the at least one protein of said first group being significantly higher and/or the level of the at least one protein of said second group being significantly lower than the level of said protein(s) in the liver or body fluid of subjects without cancer associated with increased activity of c-myc is indicative of increased c-myc activity in the subject, and optionally further comprising the above-mentioned screening method to identify a compound for modulating the increased c-myc activity in the liver of the subject.

The invention also concerns a method, in particular the aforementioned method, for predicting the response of a liver cell dysplasia or HCC patient to the administration of a c-myc activity modulator, wherein the level of at least one protein selected from said first group of proteins being significantly higher and/or the level of at least one protein selected from said second group of proteins being significantly lower in the liver tissue or body fluid of said patient than the level of said protein(s) in the liver tissue or body fluid of subjects without liver cell dysplasia or HCC associated with increased activity of c-myc is indicative that the patient will respond therapeutically to a method of treating cancer comprising administering a c-myc activity modulator.

In a preferred embodiment, the methods of the invention are implemented by performing an immunoassay, such as an enzyme immunoassay (EIA), a radio immunoassay (RIA) or a fluorescence immunoassay (FIA) may be, in particular by using the kit according to the invention and/or by performing an immunohistochemical analysis or a western blot.

Preferably, at least one antibody specific for a protein selected from the group consisting of Phosphatidylinositol-glycan-specific phospholipase D, Alpha-fetoprotein, Antithrombin 3, Apolipoprotein E, Apolipoprotein M, Fibrinogen beta-chain, Haptoglobin, Paraoxonase 1, Retinal binding protein, Serum amyloid P-component, and Transthyretin and/or at least one antibody specific for a biomarker selected from the group consisting of Afamin, Glutathione peroxidase 3, Hemopexin, Major urinary protein, and Serine protease inhibitor A3K, is used for the immunoassay and/or reagents effective to detect said biomarker(s) in a serum sample, such as a blocking buffer for reducing unspecific antibody binding or an enzyme substrate for imaging enzyme labelled antibodies may be, is used for the immunoassay.

In particular it is preferred if an immunohistochemical analysis and/or a western blot is performed for determining the presence or concentration of at least one of said proteins, and wherein preferably at least one of said antibodies is used, and/or wherein dysplastic or malignantly transformed cells isolated from liver tissue by laser microdissection are used.

In another preferred embodiment, the method is implemented by performing a peptide mass fingerprinting, in particular by using the kit described herein.

In one embodiment, the methods according to the invention comprise the steps of
adding lysis buffer to a sample, preferably a serum sample or a liver tissue sample, isolated from a subject suffering from or being susceptible to liver cell dysplasia or HCC;
separating the proteins of the lysed serum sample by 2D gel electrophoresis;
excising from the gel at least one 2-D spot containing a differentially regulated protein;
adding digesting buffer, preferably a buffer containing trypsin, to the at least one excised sample;
determining the identity of the protein by analyzing the digested 2-D spot by mass spectrometry.

According to the invention, the identity, or the presence and/or the concentration, respectively, of the proteins Polymeric immunoglobulin receptor, Phosphatidylinositol-glycan-specific phospholipase D, Afamin, Alpha-fetoprotein, Antithrombin 3, Apolipoprotein E, Apolipoprotein M, Fibrinogen beta-chain, Haptoglobin, 1, Retinol binding protein, Serum amyloid P-component, Transthyretin, Glutathione peroxidase 3, Hemopexin, Major urinary protein, and Serine protease inhibitor A3K may be determined by determining the presence or concentration of fragments, being 7-24 amino acid residues in length, of said proteins, in particular by determining the presence or concentration of at least one peptide according to Table 2, preferably in a tissue or body fluid sample, which may have been further processed, such as by 2-DE, and wherein a protease, preferably trypsin, has been added to said preferably further processed sample.

In particular, a method according to the invention is preferred, wherein peptide mass fingerprinting is performed, preferably based on mass spectrometry with 2D tryptic digested spots selected by recognition and identified by MALDI-TOF, ESI-TOF or 1TMS, for determining the presence or concentration of the selected protein, preferably in the body fluid or tissue.

In another embodiment, a method according to the invention is preferred, wherein the expression of the gene coding for the selected protein is determined by means of a PCR, preferably a RT-PCR and/or a quantitative real time PCR, for determining the presence or concentration of said protein, preferably in the sample of the tissue isolated from of the liver.

The invention is further directed to a kit for the use in qualifying the c-myc activity in a subject suffering from or being susceptible to cancer or dysplasia, in particular liver cell dysplasia or HCC, preferably for use in a method according to the invention, in particular for predicting or monitoring the response of a liver cell dysplasia or HCC patient to a method of treating cancer comprising administering a c-myc activity modulator, wherein the kit comprises at least one standard indicative of the level of a protein selected from the group consisting of Polymeric immunoglobulin receptor, Phosphatidylinositol-glycan-specific phospholipase D, Afamin, Alpha-fetoprotein, Antithrombin 3, Apolipoprotein E, Apolipoprotein M, Fibrinogen beta-chain, Haptoglobin, Paraoxonase 1, Retinol binding protein, Serum amyloid P-component, Transthyretin, Glutathione peroxidase 3, Hemopexin, Major urinary protein, and Serine protease inhibitor A3K, in the liver or in a serum sample, of normal individuals or in the liver or serum of individuals having liver cell dysplasia or HCC associated with increased c-myc activity, and/or at least one preferably synthetic fragment, being 7-24 amino acids in length, of at least one of said proteins, in particular at least one of the peptides according to Table 2, and/or at least one antibody specific for said protein(s), and/or at least one primer pair for determining the mRNA coding for the protein, and instructions for the use of the kit.

The invention also concerns the use of at least one biomarker selected from the group consisting of the proteins Polymeric immunoglobulin receptor, Phosphatidylinositol-glycan-specific phospholipase D, Afamin, Alpha-fetoprotein, Antithrombin 3, Apolipoprotein E, Apolipoprotein M, Fibrinogen beta-chain, Haptoglobin, Paraoxonase 1, Retinol binding protein, Serum amyloid P-component, Transthyretin, Afamin, Glutathione peroxidase 3, Hemopexin, Major urinary protein, Serine protease inhibitor A3K, or of at least one antibody directed against said at least one biomarker, in the diagnosis, prognosis and/or treatment monitoring of cancer or dysplasia, in particular of HCC or liver cell dysplasia.

Preferably, an appropriate amount of the at least one biomarker is used, in particular an amount for manufacturing a reference, more particular for manufacturing a reference comprising a reference level of said at least one biomarker, such as the level of said at least one biomarker in a sample of a normal healthy subject or the level of a said at least one biomarker in a sample of a patient suffering from HCC or having liver cell dysplasia may be.

In particular, at least one of said biomarkers and/or at least one antibody directed against said at least biomarker, is used for monitoring the therapeutic treatment of a patient suffering from HCC or having liver cell dysplasia, in particular the treatment with a chemotherapeutic drug, preferably with an antineoplastic chemotherapy drug, or with a chemopreventive drug.

Further, a composition for diagnosing or treatment monitoring of dysplasia or cancer, in particular of liver cell dysplasia or HCC, associated with increased c-myc activity in a patient, preferably by an in vitro body fluid analysis, is provided according to the invention, comprising an effective amount of at least one biomarker selected from the group consisting of the proteins Polymeric immunoglobulin receptor, Phosphatidylinositol-glycan-specific phospholipase D, Afamin, Alpha-fetoprotein, Antithrombin 3, Apolipoprotein E, Apolipoprotein M, Fibrinogen beta-chain, Haptoglobin, Paraoxonase 1, Retinol binding protein, Serum amyloid P-component, Transthyretin, Afamin, Glutathione peroxidase 3, Hemopexin, Major urinary protein, and Serine protease inhibitor, or of a preferably synthetic fragment, being 7-24 amino acids in length, of at least one of said proteins, in particular of at least one of the peptides according to Table 2, or comprising at least one antibody directed against said at least one biomarker, in particular for use in diagnosing or treatment monitoring of dysplasia or cancer, preferably of liver cell dysplasia or HCC, associated with increased c-myc activity in a patient.

Said composition is preferably used for the production of a diagnostic agent, in particular of a diagnostic standard for body fluid analysis, or, more particular, for the production of a diagnostic agent for qualifying the c-myc activity in a patient suffering or being susceptible to cancer or for classifying a patient suffering from or being susceptible to HCC.

Within this context, said composition is particularly used for the production of a diagnostic agent for predicting or monitoring the response of a cancer patient to a method of treating cancer comprising administering a c-myc activity modulator, e.g. an inhibitor of c-myc/max dimerization.

In yet another preferred embodiment, said composition further comprises an effective amount of a protease, in particular of trypsin, thus enabling a further enhancement of the system sensitivity.

Said composition, in particular the protease digest thereof, may be preferably used for producing a vaccine for the immunization of an animal in order to produce polyclonal antibodies specific for the at least one biomarker.

Within the context of the invention, also a method of qualifying the c-myc activity in a patient suffering or being susceptible to cancer or for classifying a patient suffering from or being susceptible to HCC is provided, comprising determining in a body fluid sample of a subject suffering from or being susceptible to cancer at least one biomarker selected from the first group consisting of the proteins Polymeric immunoglobulin receptor, Phosphatidylinositol-glycan-specific phospholipase D, Alpha-fetoprotein, Antithrombin 3, Apolipoprotein E, Apolipoprotein M, Fibrinogen beta-chain, Haptoglobin, Paraoxonase 1, Retinol binding protein, Serum amyloid P-component, and Transthyretin, and/or at least one biomarker selected from the second group consisting of the proteins Afamin, Glutathione peroxidase 3, Hemopexin, Major urinary protein, and Serine protease inhibitor A3K, wherein the body fluid level of the at least one biomarker of said first group being significantly higher and/or the body fluid level of the at least one biomarker of said second group being significantly lower than the level of said biomarker(s) in the body fluid of subjects without cancer associated with increased activity of c-myc is indicative of induced c-myc activity in the subject.

In one embodiment, said method is preferably used for predicting the response of a cancer patient to a method of treating cancer comprising administering a c-myc activity modulator, wherein the body fluid level of the at least one biomarker of said first group being significantly higher and/or the body fluid level of the at least one biomarker of said second group being significantly lower than the level of said biomarker(s) in the body fluid of subjects without cancer associated with increased activity of c-myc is indicative that the subject will respond therapeutically to a method of treating cancer comprising administering a c-myc activity modulator.

In another embodiment, said method is used for monitoring the therapeutically response of a cancer patient to a method of treating cancer comprising administering an c-myc activity modulator, wherein the body fluid level of the at least one biomarker of said first group before and after the treatment and/or the body fluid level of the at least one biomarker of said second group before and after the treatment is determined, and a significant decrease of said body fluid level(s) of the at least one biomarker of said first group and/or a significant increase of said body fluid level(s) of the at least one biomarker of said second group after the treatment is indicative that the subject therapeutically responds to the administration of the c-myc activity modulator.

Moreover, a procedure to screen for and to identify drugs against cancer associated with an increased c-myc activity is provided, comprising determining in a body fluid sample of a transgenic cancer mouse being treated with a compound to be tested, in particular of a mouse whose genome comprises a non natural c-myc sequence, at least one biomarker selected from the first group consisting of the proteins Polymeric immunoglobulin receptor, Phosphatidylinositol-glycan-specific phospholipase D, Alpha-fetoprotein, Antithrombin 3, Apolipoprotein E, Apolipoprotein M, Fibrinogen beta-chain, Haptoglobin, Paraoxonase 1, Retinol binding protein, Serum amyloid P-component, and Transthyretin, and/or at least one biomarker selected from the second group consisting of the proteins Afamin, Glutathione peroxidase 3, Hemopexin, Major urinary protein, and Serine protease inhibitor A3K, wherein the body fluid level of the at least one biomarker of said first group being significantly lower and/or the body fluid level of the at least one biomarker of said second group being significantly higher than the level of said biomarker(s) in the body fluid of an untreated transgenic cancer mouse is indicative of the therapeutic effect of said compound as a c-myc activity modulator.

For implementing the methods or uses according to the invention, in particular for determining the presence, concentration or expression of a protein, it may be favourable to use one of the following methods—PCR, in vitro translation, RT-PCR, gel electrophoresis, Western Blot, Northern Blot, Southern Blot, ELISA, FACS measurement, chromatographic isolation, UV microscopy, immunohistochemistry, screening of solid phase bound molecules or tissues, mass spectrometra, and/or biosensory investigation—whereby by amplification, isolation, immobilization and/or detection and/or by combinations of thereof a particularly simple conversion of the methods or according to invention is made possible for the examined sample, in particular if furthermore a statistic analysis is accomplished.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

Background & Aims: The incidence of hepatocellular carcinoma (HCC) is on the rise with hundreds of thousands of new cases each year. Early detection of disease will improve patient outcome but so far only a few serum biomarkers are available. Based on findings with a transgenic disease model, we aimed for an identification of novel serum biomarkers of human HCC. Methods: The serum proteome of healthy and transgenic mice was analyzed by 2-DE at pH 3-10 and 4-7, respectively. More than 4500 tryptic in-gel digests derived from thirty-six 2-DE gels were analyzed by MALDI TOF/TOF peptide mass fingerprinting. Regulated proteins were also investigated in n=20 sera and n=12 tissues of HCC patients by Western immunoblotting and immunohistochemistry. Results: From 2-DE analysis, 321 serum protein spots were >2-fold regulated, of which 143 were up- and 178 down-regulated. Notably, 17 proteins were significantly regulated of which 3 are novel biomarker candidates, namely the phosphatidylinositol-glycan-specific phospholipase D, the polymeric immunoglobulin receptor and afamin, a new member of the albumin family. Furthermore, proteins known to be up-regulated in solid cancers such as alpha-fetoprotein, antithrombin-III, fibrinogen and haptoglobin were up-regulated in serum of HCC mice as well, while glutathione peroxidase 3 and serine protease inhibitor A3K were down-regulated, albeit at different levels. The up-regulation of apoliproteins E, paraoxonase 1, retinol binding protein, serum amyloid P and transthyretin were additionally confirmed in human HCC tissue. Conclusions: Translational research enabled an identification of novel serum and tissue biomarkers of HCC worthy for in depth clinical validation.

Here we report our efforts to map the serum proteome of a transgenic disease model where targeted expression of c-Myc to liver resulted in HCC. Based on 2-DE and MALDI-TOF/TOF MS we identified 17 regulated proteins (excluding isoforms) when the serum of healthy and HCC bearing mice were compared. Some of the regulated proteins were further validated by Western immunoblotting (WB) and immunhistochemistry (IHC) of serum and tumor tissue of patients and mice diagnosed with HCC. Overall, we report three novel serum biomarkers of HCC and confirmed their regulation in HCC of human and mouse origin.

### INTRODUCTION

### Materials and Methods

### Transgenic Mouse Model and Mouse Serum

All animal work followed strictly the Public Health Service (PHS) Policy on Human Care and Use of Laboratory Animals. Formal approval to carry out animal studies was granted by the ethical review board of the city of Hannover (Germany). Transgenic mice were the kind gift of Dr. Dalemans [11]. They were maintained as hemozygotes in the C57/BI6 black round. The transgene was verified by PCR using the forward primer: 5′-CACTGCGAGGGGTTCTGGAGAGGC-3′ and the reverse primer: 5′-ATCGTCGTGGCTGTCTGCTGG-3′ and the following assay conditions: 15 min 95° C., 1 min 60° C., 1 min 70° C., 1 min 95° C., 31 cycles. Six healthy non-transgenic (C57B16), n=6 HCC bearing mice aged between 10-12 months and n=3 transgenic mice without cancer aged between 5.5-6.5 months were kept individually with food and water given ad libitum. Blood serum was collected from the vena cava and allowed to clot for 2 hours at room temperature. The clotted material was removed by centrifugation at 3000 rpm for 15 min. The obtained sera were immediately frozen in liquid nitrogen and stored at −80° C. until further analysis. Serum protein concentration was determined by the Bradford assay (Bio-Rad) and ranged between 108 to 128 μg/μl for non-transgenic, 128 to 145 μg/μl for AAT- c-Myc transgenic mice aged between 5.5-6.5 months and in the case of HCC bearing mice (aged between 10-12 months), the serum concentration ranged between 135 to 160 μg/μl.

### Patient Characteristics and Human Serum

Characteristics of patients are given in Supplementary Material 1. Specifically, human tumor tissue blocks from group A patients (IHC analysis) were provided by Dr. Ferdinand Hofstädter, Institute of Pathology, University of Regensburg (Germany); characteristics in group B are of patients whose sera were used for WBs analysis. Serum was obtained as described above and stored immediately at 80° C. Protein concentration (BioRad assay) ranged between 90-185μg/μl (healthy individuals) and between 125-252μg/μl (HCC patients).

### 2-DE and Image Analysis

For gel electrophoresis a total of 500 μg/gel was loaded and subjected to two-dimensional electrophoresis (2-DE) as described previously [12]. Briefly, proteins were focused by their isoelectric behavior by use of IPG strips of the same lot. Gels were run at the pH of 3-10 and 4-7, respectively in the first and second dimension. Then, protein spots were recorded as digitalized images using a high-resolution scanner (Expression 10000XL, Epson).

2-D gel images were analyzed with the PDQuest™ software (version 8.0.1; Bio-Rad) with the Spot Detection Wizard. Scanned gel images were further processed to remove background noise as to enable automatic spot detection. Protein spots were considered regulated if identified in at least 3 out of 6 serum protein maps. Note, 2-DE was done in triplicate for each individual serum sample giving rise to a total of 36×2-DE gels. The spot intensity was calculated as normalized values with LOESS (Local Regression Model); data were expressed in ppm. Quantification was done by means of intensity (total intensity/pixel number) with the PDQuest™ quantity tool and spot variation was estimated by quantity and coefficient of variation (CV) (see Table 1 and Supplementary Material 2). Only spots showing at least two-fold difference were accepted as significantly changed (100%) [13]. For between group comparisons the Student's t-test and the Mann-Whitney U-test was used, the latter test being more robust against violations of normality. P-values<0.05 were considered as significant [14]. Additionally, partial least squares (PLS) multivariable test was applied to test for significance (see Table 2).

### MALDI-TOF/TOF Mass Spectrometry

Tryptic digested peptides were spotted onto a 600 μm/384 well AnchorChip™ sample target (Bruker) and PMF and tandem MS were done with an Ultra Flex™ II MALDI-TOF/TOF (Bruker) equipped with a smartbeam™ laser. Specifically, for sample/matrix preparations we used the α-cyano-4-hydroxycinnamic acid (CHCA) and 2,5-dihydroxybenzoic acid (DHB) matrices; CHCA was saturated in 97% Acetone/0.1% TFA solution; DHB was dissolved in 30% ACN/0.1% TFA solution (5 mg/ml). The matrix-analyte preparations were loaded by the thin layer and the matrix layer (ML) method as described recently [15]. External peptide calibration standards were used to calibrate the instrument (Bruker). Additionally, internal calibration was achieved using trypsin autolysis products (m/zₛ 1045.564, 2211.108 and 2225.119) resulting in a mass accuracy of <50 ppm. Based on initial data, ion precursors were selected for tandem MS data acquisition. Trypsin autolysis products, tryptic peptides of human keratin and matrix ions were automatically discarded by ProteinScape™ (Bruker).

Then peptide masses were searched against the Swiss-Prot database (download 2006) employing the MASCOT version 2.0 (Matrix Science) and taking carbamidomethyl modifications of cysteines and possible oxidation of methionine into account but allowing one missed cleavage [For further details see ref 12].

### Western Immunoblotting

Serum protein extracts were subjected to Western immunoblotting using polyclonal goat and/or rabbit antibodies against alpha-fetoprotein, apolipoprotein E, retinol binding protein, serum amyloid P in a 1:200 dilution and major urinary protein and transthyretin in a 1:100 dilution (Santa Cruz Biotechnology) and apolipoprotein M in a dilution of 1:10000 (BD Bioscience). Notably, total protein extracts from HepG2 human hepatoma cells and Hela total cell extracts served as positive control. For human serum, stock solutions of series Prestige Antibodies® (Swedish Human Protein Atlas (HPA) Program-www.proteinatlas.org-) of rabbit origin were purchased from Sigma-Aldrich (rabbit anti-human AFM, GPLD1, PIGR and PON1) and diluted in the range 1:500-1:2000. Detection was based on the ECL (PerkinElmer) and WesternDot™ 625 (Invitrogen) systems according to the manufactory recommendations. Images were recorded on the Kodak ds (IS 440CF) or alternatively with the PharosX (BioRad) fluorescence detector in the case of the Qdot® 625 nanocrystals of streptavidin conjugates. Band semi-quantitation was achieved with the QuantityOne® 1D Analisys software (version 4.6.1; BioRad) using local background subtraction, while data were expressed relative to alpha-tubulin (T/C 1.01±.025) that served as a house-keeping protein (Supplementary Material 3).

### Immunohistochemistry

Healthy livers of n=5 individuals aged between 53 and 83 and n=12 cases of HCC aged between 26 and 77 were analyzed (Supplementary Material 1). Each tumor section (2-4 μm in thickness) was de-paraffinized with Roti-Histol and dehydrate in a descending alcoholic solution according to standard protocols. To unmask the antigens the sections were immersed in citrate buffer and placed in an autoclave for 15 minutes. Endogenous peroxidase activity was blocked with a 3% hydrogen peroxide/methanol peroxidase blocking solution for 30 minutes. Then, the sections were washed with water and incubated with a solution containing the primary polyclonal antibody either against apolipoprotein E or serum amyloid P (at dilution 1:200); staining for hemopexin, paroxonase 1 and major urinary protein was at 1:30. In the case of c-Myc, glutathione peroxidase 3, retinol binding protein and prealbumin a1:50 dilution of primary antibody was used and incubated for 45 minutes. After a further washing step a streptavidin horseradish peroxidase detection kit (Envision . DAKO) was employed to visualize the protein of interest as recommended by the manufacturer. Harris Hematoxylin was used as a counter-stain. The specificity of the reaction was further validated by use of mouse immunoglobulin G instead of the primary antibody (Supplementary Material 4).

### Results

The histopathology of liver of non-transgenic and transgenic mice at early and advanced stages of tumor growth is reported in detail elsewhere (Hunecke and Borlak 2009, submitted). Notably, histopathology confirmed highly as well as less differentiated HCC in 10-12 months old mice. Supplementary Material 5 depicts images of healthy and tumor livers.

### 2-DE Gel Imaging of Serum Proteins

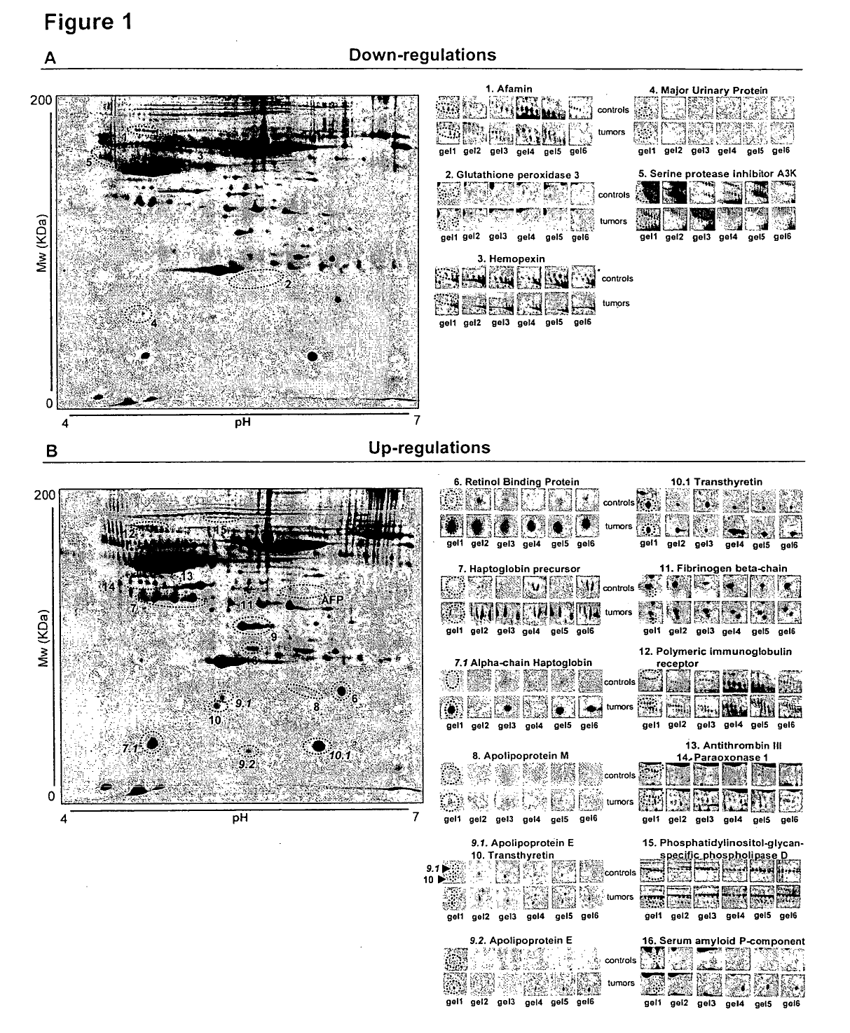

Serum protein extracts from n=6 healthy non-transgenic and n=6 tumor bearing mice were loaded in triplicate on IPG strips at pH 4-7 and 3-10, respectively. On average, 400 and 600 spots were detected by 2-DE. Images were cropped from scanned gels and used for quantification and statistical analyses. Since most of the informative spots were concentrated in the acidic part of the 2-D gels, proteins were specifically evaluated at the pH range 4-7. Data obtained at pH 3-10 are given in Table 1 and Supplementary Material 2. Amongst the different gels, a coefficient of variation of 37% was calculated but this agrees well with previous studies reported for 2-DE gels [16,17]. Notably, representative images of regulated serum proteins are depicted in FIG. 1.

### Peptide Mass Fingerprinting by MALDI-MS and Tandem MS

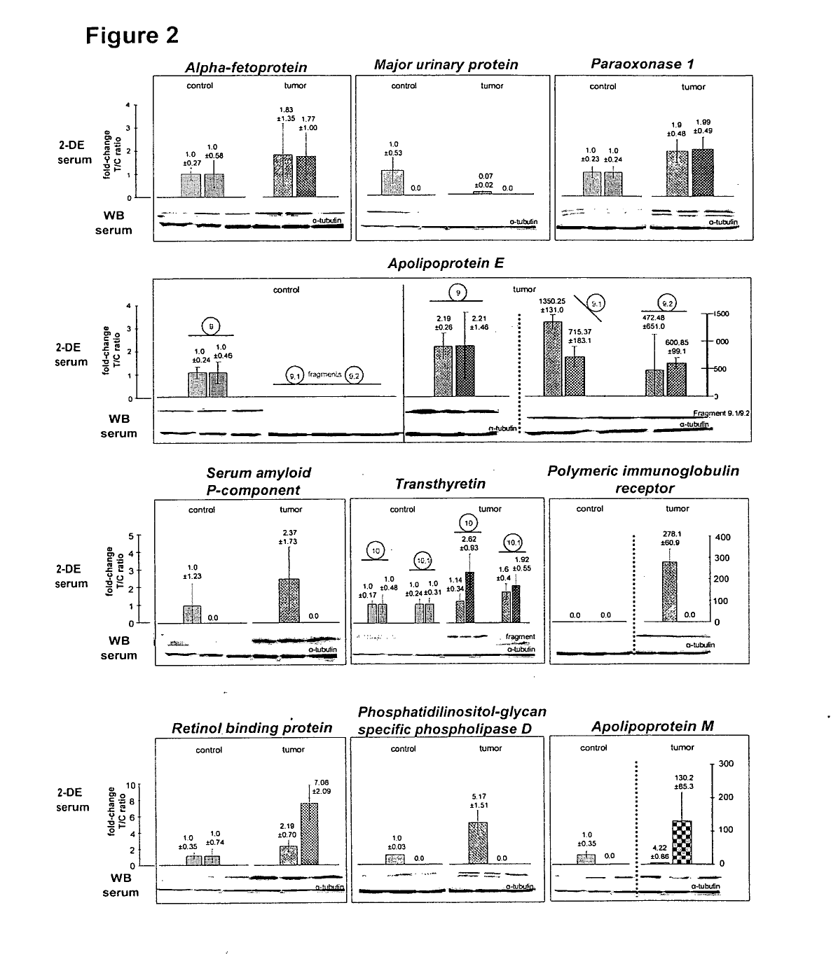

We applied MALDI-TOF/TOF-MS to obtain peptide mass fingerprints (PMF) and peptide fragmentation fingerprints (PFF) of regulated proteins [12,15]. As shown in Table 1, at least 6 peptides were matched to a protein and almost all PMFs are of high sequence coverage. Proteins were further analyzed by MALDI TOF tandem MS of the parent ion (Table 2). An example is depicted in Supplementary Material 6. Identification of both the precursor and the alpha-chain of haptoglobin could be confirmed by MS/MS analyses. Fragmentation of the parent ion at m/z 980 identified spot 7 (see FIG. 1B) as haptoglobin precursor, while the peak at m/z 1679 was the haptoglobin alpha chain (see spot 7.1). Likewise, apolipoprotein E was traced back to spots 9.1 and 9.2 respectively (FIG. 1B). Specifically, we identified peptides of apoE of amino acids 87-144 with an ion score of up to 105 and observed fragments of this lipoprotein in tumor bearing mice only (FIGS. 1B and 2).

In the same way, isoforms of transthyretin were visualized by 2-DE and identified by MALDI-MS. For the spots 10 and 10.1 (FIG. 1B) a high MASCOT score (87 and 133, respectively) and a high sequence coverage (51 and 67%) could be obtained (Tables 1 and 2). Thus, tandem MS yielded ions at m/z 869, 1382 and 2438 belonging to AA 56 to 123 of transthyretin (see spot 10.1); whereas AA 56 to 147 of transthyretin were traced back to spot 10.

We identified major urinary protein with a MASCOT score of 184 and sequence coverage of 74% and matched 13 peptides to this protein even though the corresponding spots on the 2-D gels were hardly visible (see FIG. 1A).

### Western Immunoblotting and Immunohistochemistry of Regulated Proteins

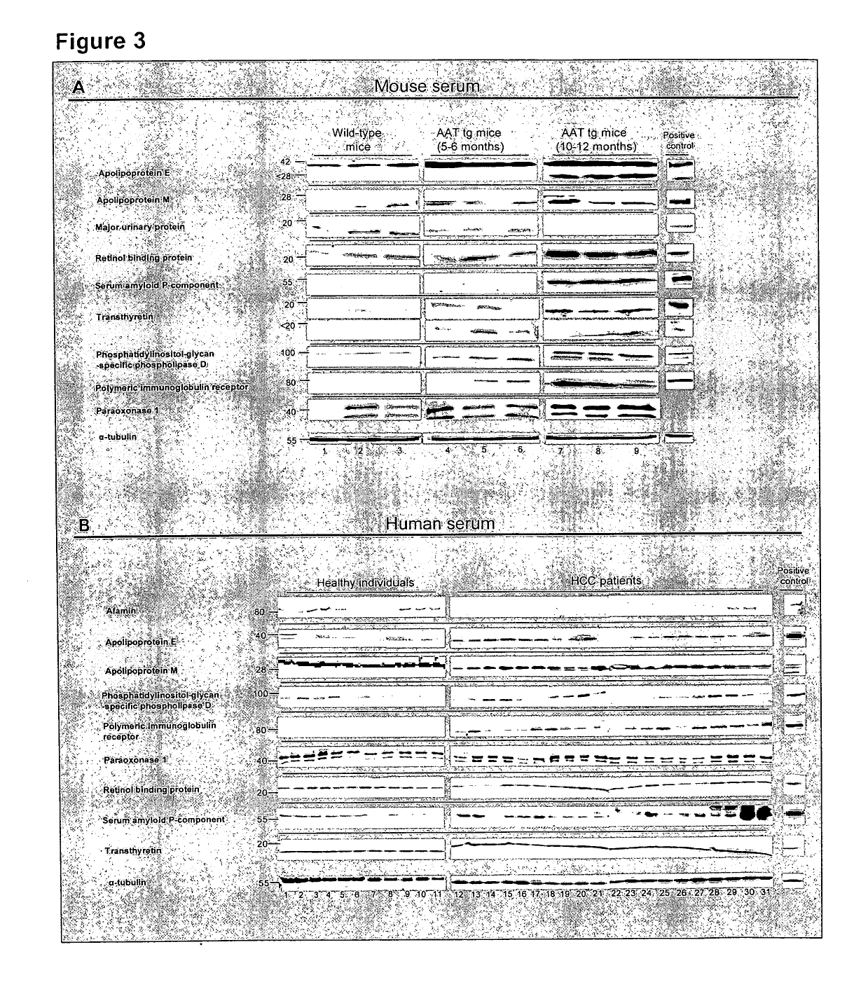

Some proteins were selected for further validation by Western immunoblotting (WB). Basically there was good agreement between the PDQuest™ image analyses and the WB results (see Table 2, FIG. 2). We compared results at different stages of disease in mouse, e.g. dysplasia and subsequent HCC, and noted that major urinary protein was clearly detectable even at the early stage of disease while transthyretin increased expression in disease progression. No signal at low Mw for apolipoprotein E was observed when transgenic mice at early stages of disease were analyzed (FIG. 3A).

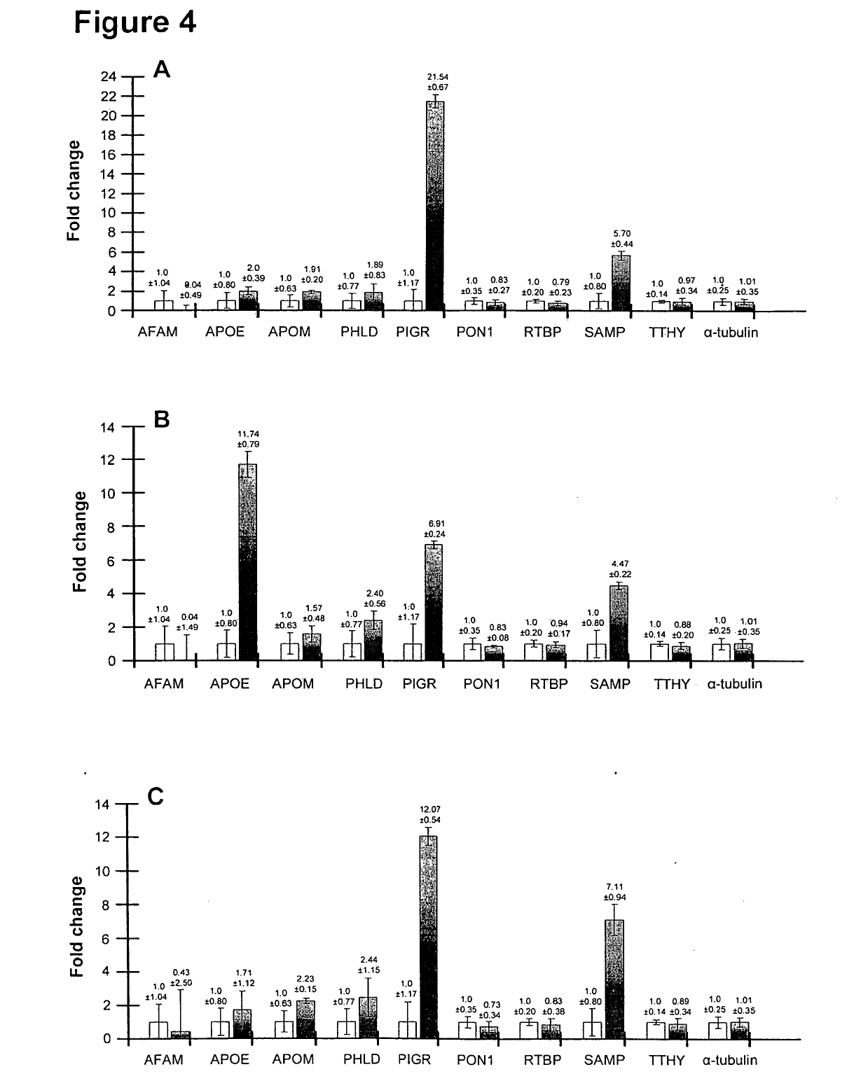

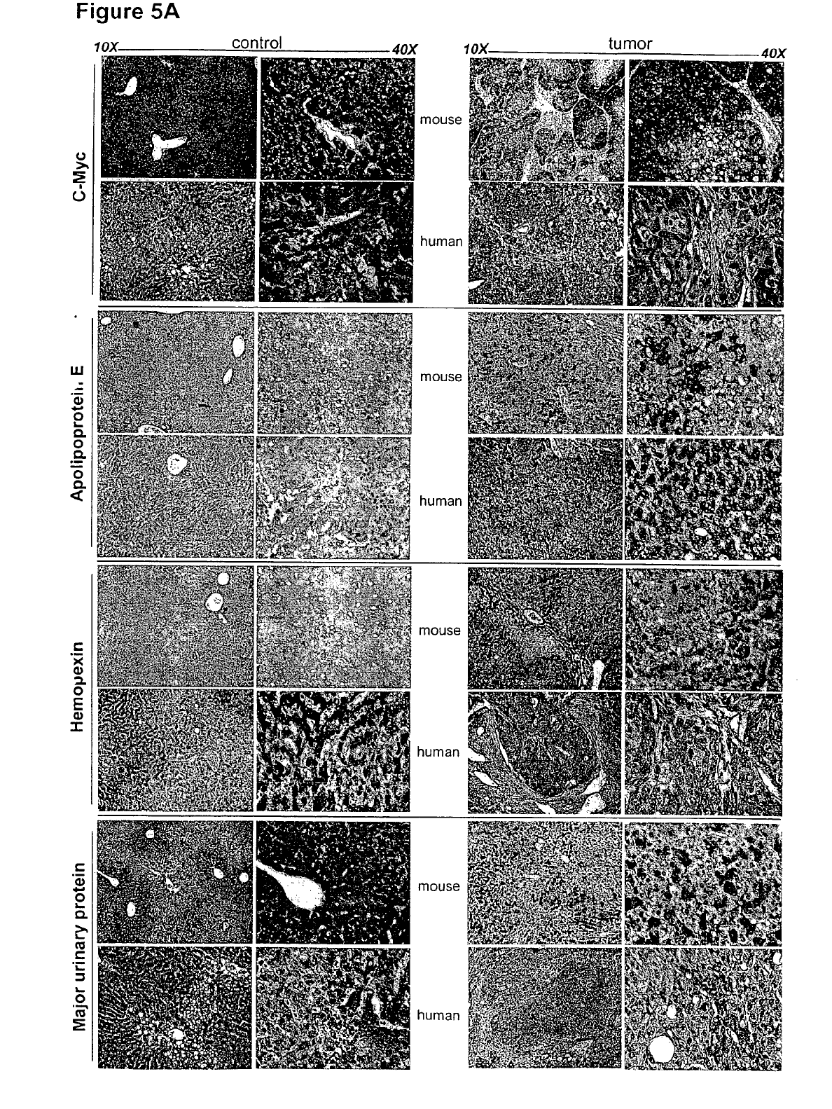

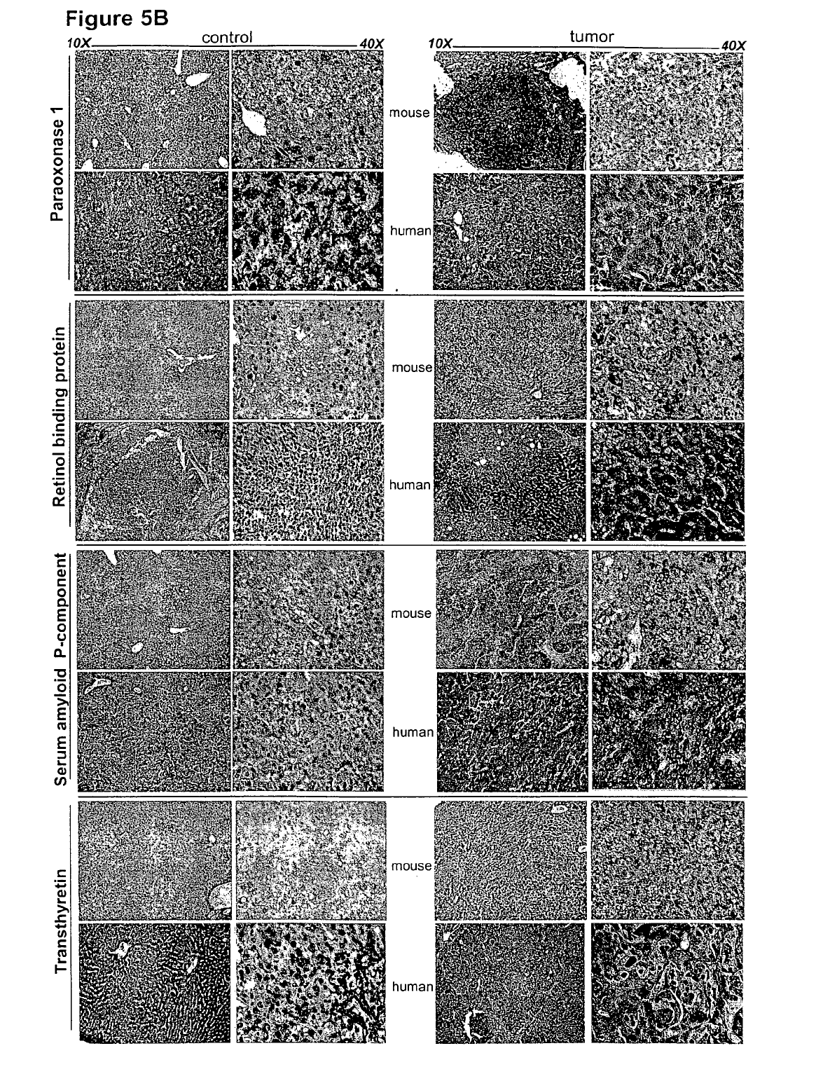

Serum samples from n=11 healthy individuals and n=20 HCC patients (10 males+10 females, data in Supplementary Material 3) were analyzed in parallel. As shown in FIG. 3B, expression of afamin (basically absent in all but 2 tumor patients), apolipoproteins E and M, phosphatidylinositol-glycan-specific-phospholipaseD, polymeric-immunoglobulin-receptor, as well as serum amyloid P-component were regulated similarly in mice and human serum samples, thereby enabling translational research for an identification of candidate biomarkers for HCC. Unlike findings with mice, there was no regulation for paraoxonasel, retinol binding protein and transthyretin in serum of HCC patients (see FIGS. 3B, 4A,B,C). Interestingly, PHLD resulted less up-regulated in viral cases (FIG. 4A) than in other cases (B and C sections); this can be considered as a starting-point for further experiments with a wider HCC patient group to prove PHLD as candidate biomarker for non-viral HCC. Moreover, we employed immunohistochemistry (IHC) to search for regulated proteins in human and murine liver tissues. FIGS. 5A and 5B depict examples of IHC applied to mouse and human HCC. The full set of IHC is given in Supplementary Material 4. With the exception of hemopexin and major urinary protein, there was agreement when regulation of proteins in serum and tumor tissue was compared. Additionally, in the case of c-Myc and to a lesser extent with retinol binding protein nuclear staining was predominant, while for the remaining IHC cytosolic staining was obvious.

### Discussion

The search for serum cancer biomarkers is the subject of intense research. Here we report studies with a transgenic mouse model of HCC that enabled translational research and an identification of novel biomarkers of HCC worthy for clinical validation. Specifically, an increased resolution at the acidic part of 2-DE gels was achieved that proofed to be particularly useful for an identification of 50-100 KDa proteins. As we discussed earlier [12] the zooming of 2-D gels allowed better resolution of spots thereby avoiding co-migration of proteins. Thus, high molecular weight proteins are well resolved by trains of spots (see FIG. 1) and permitted identification of phosphatidylinositol-glycan-specific phospholipase D, polymeric immunoglobulin receptor, some isoforms of major urinary protein as well as hemopexin that were well separated and are shown to be clearly regulated (FIGS. 1A and 1B). Unlike with gels at pH 3-10, we obtained at pH 4-7 better sequence coverage, i.e., up to 74% with high peak intensity that enabled their conclusive identification by MALDI-TOF tandem MS (Table 2).

### Protein Identification by MALDI-MS and Tandem MS

As recently reported, we chose two different matrices in sequence, i.e. CHCA and DHB, according to their complementary behavior for peptide ionization [12]. Notably, as little as 100 amol were sufficient to identify proteins by MS with the DHB matrix [15]. In the case of transthyretin, we were able to characterize two isoforms with a maximum of 8 matched peptides to an entire sequence; 4 of them were fragmented and analyzed further by tandem MS. A truncated form of transthyretin lacking the first NH₂-terminal 10 amino acids had been already proposed as a biomarker of early stage ovarian cancer [18,19] while recent studies identified transthyretin Ser²⁸-G1n¹²⁸ (Mw 13 KDa) as a candidate biomarker for nephrotoxicity [20]. Here we report two forms of transthyretin as being regulated by 2-DE. This protein is involved in the transport of retinol by interacting with retinol binding protein. Furthermore, a total of 12 peptides were matched to retinal binding protein, resulting in sequence coverage of 61%. Indeed, we observed up-regulation of retinal binding protein in gels at both pH ranges from tumor-bearing mice.

We found haptoglobin to be up-regulated. This protein is an acute phase protein that has hemoglobin-binding capacity and plays a role in inflammatory reactions but also functions as an antioxidant. As depicted in FIG. 1B, a protein of Mw 8-9 was identified as haptoglobin in serum of HCC. Thus, MALDI-TOF/TOF MS and MS/MS analyses identified the N-terminal part of the haptoglobin alpha-chain. Note, recent studies confirmed regulation of alpha chain variants in ovarian cancer [21,22].

Furthermore, by tandem MS a peptide at m/z 1679 was identified that consisted of amino acids 58-72 of haptoglobin; this is in perfect agreement with the Swiss-Prot database. Thus, Y-ions and B-ions covered most of the fragments derived from MS/MS resulting in an ion score of 63 (Table 2, Supplementary Material 6).

### Biological Significance of Disease Regulated Proteins

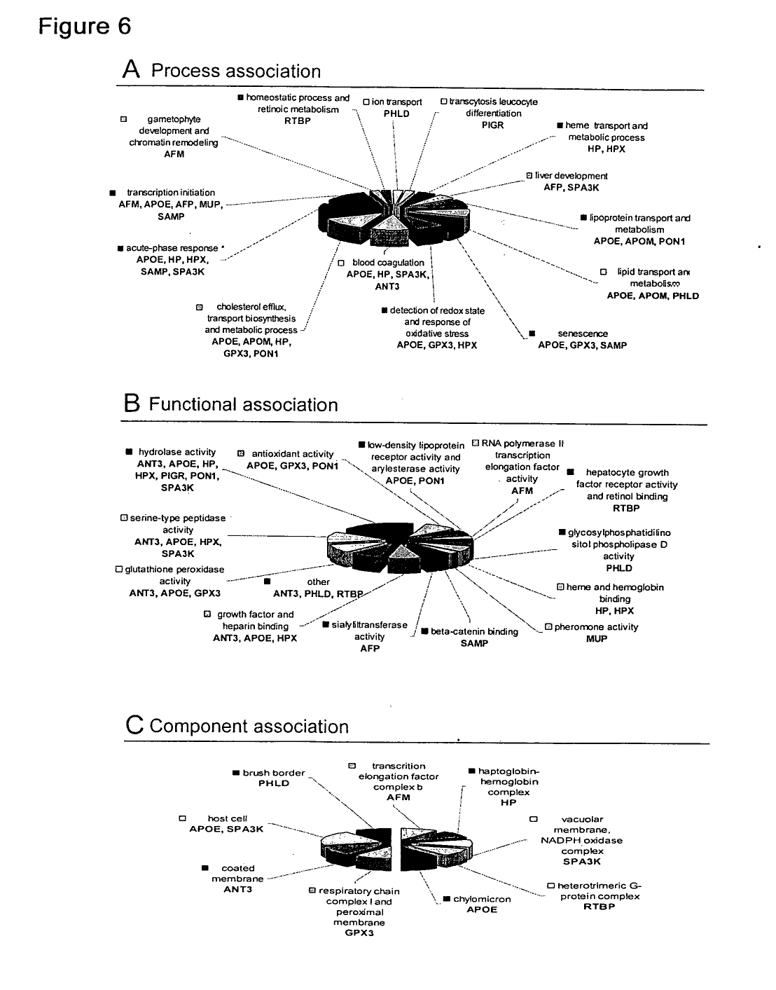

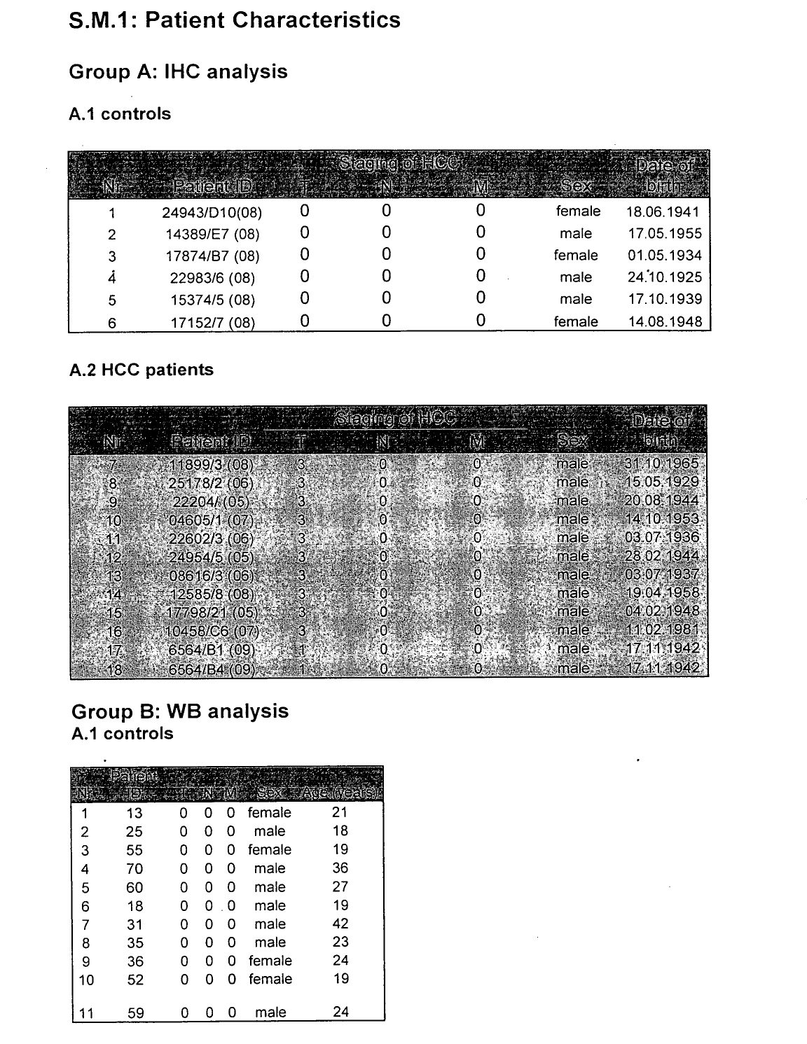

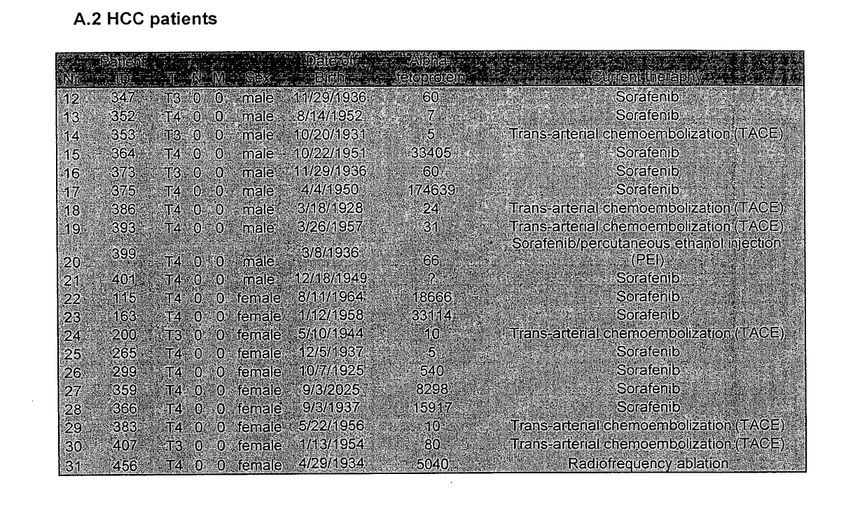

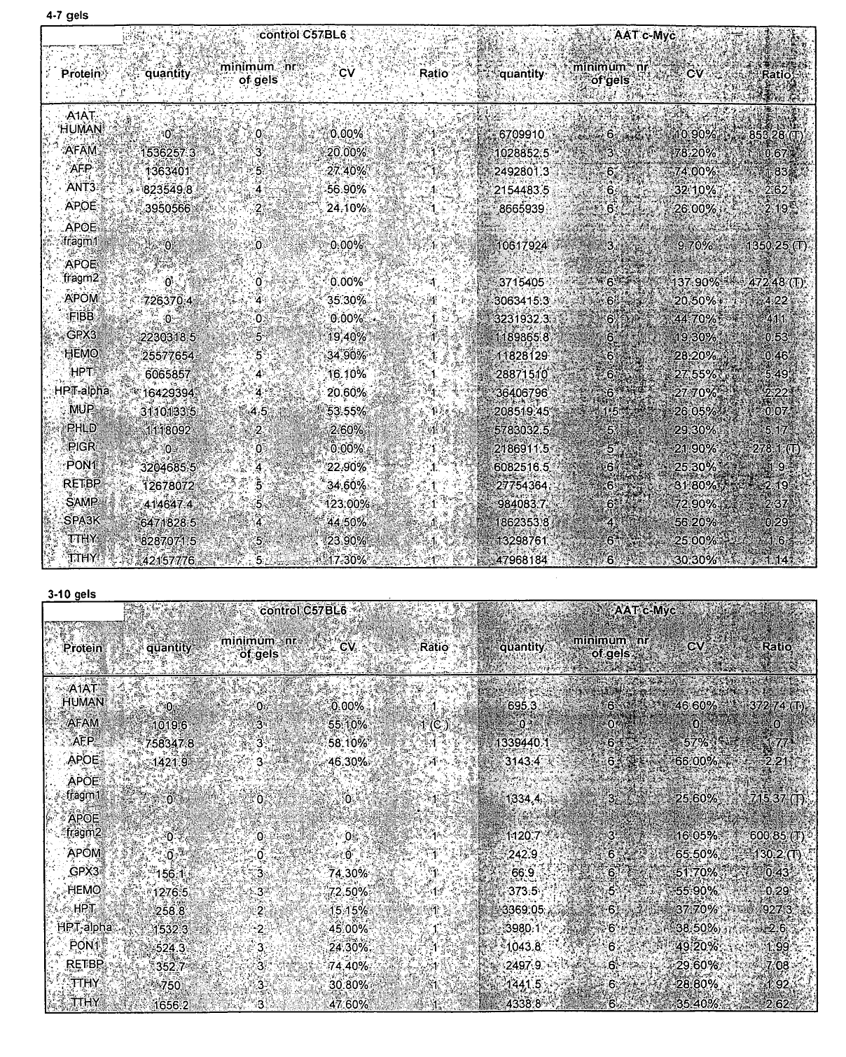

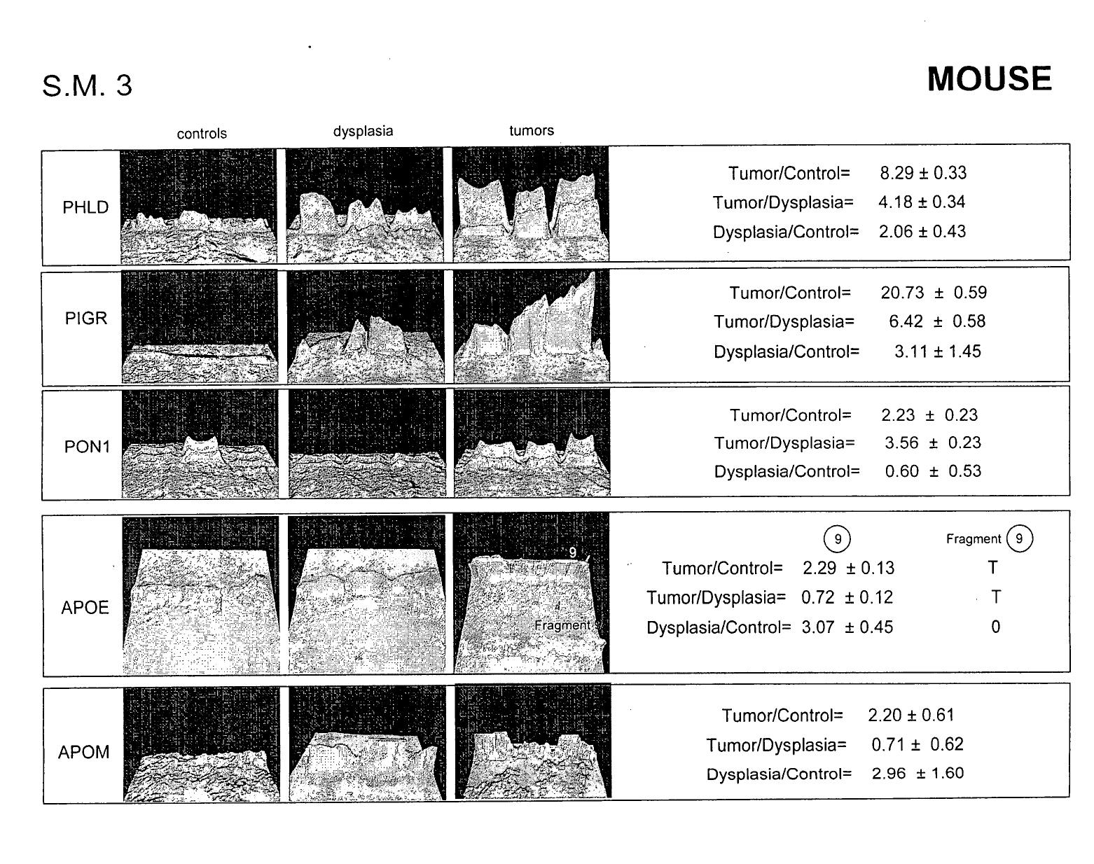

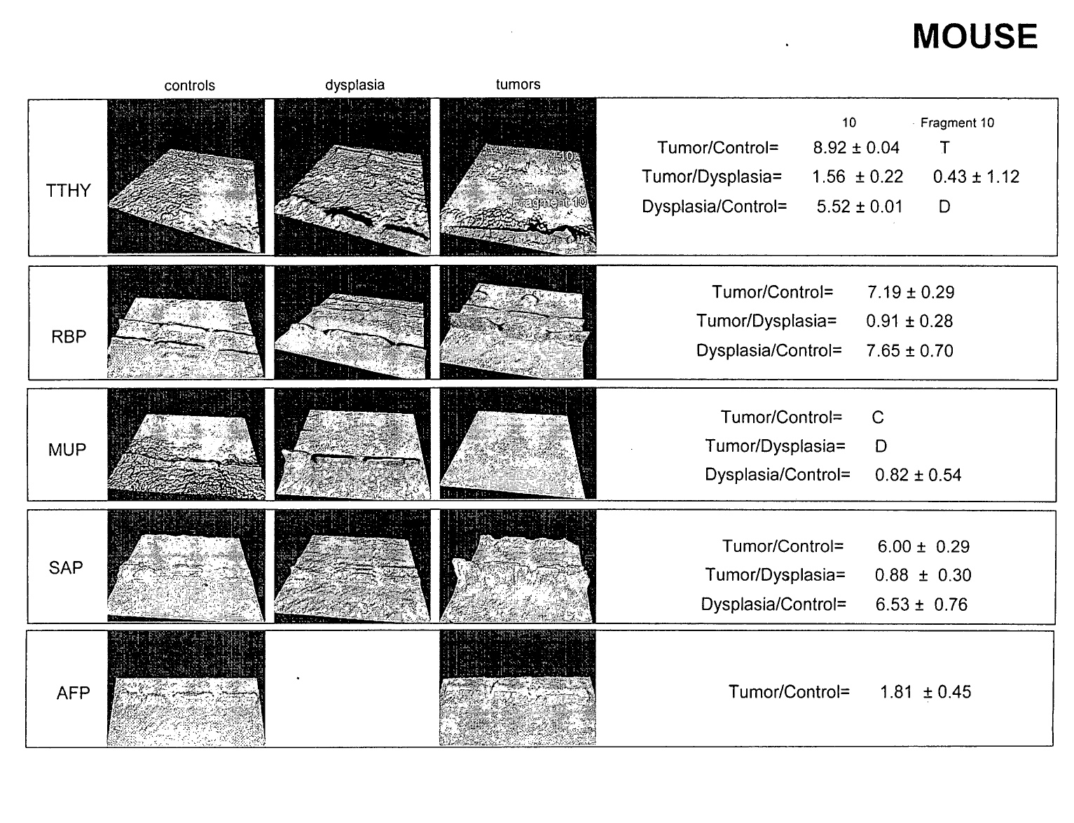

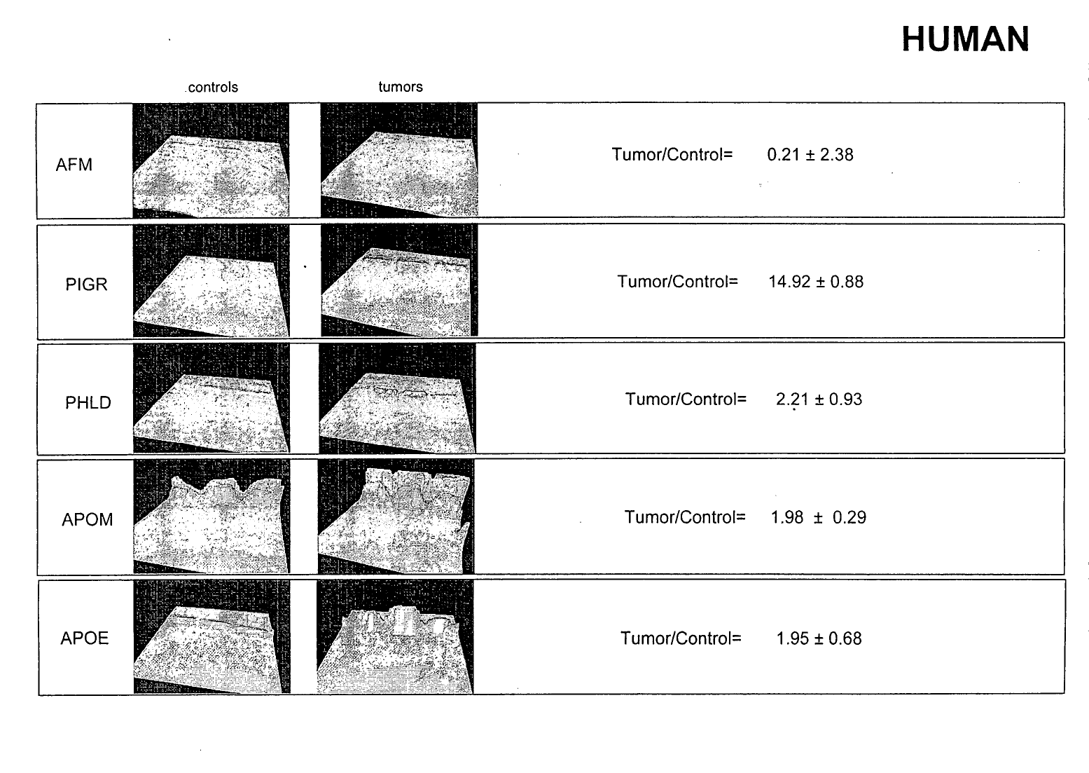

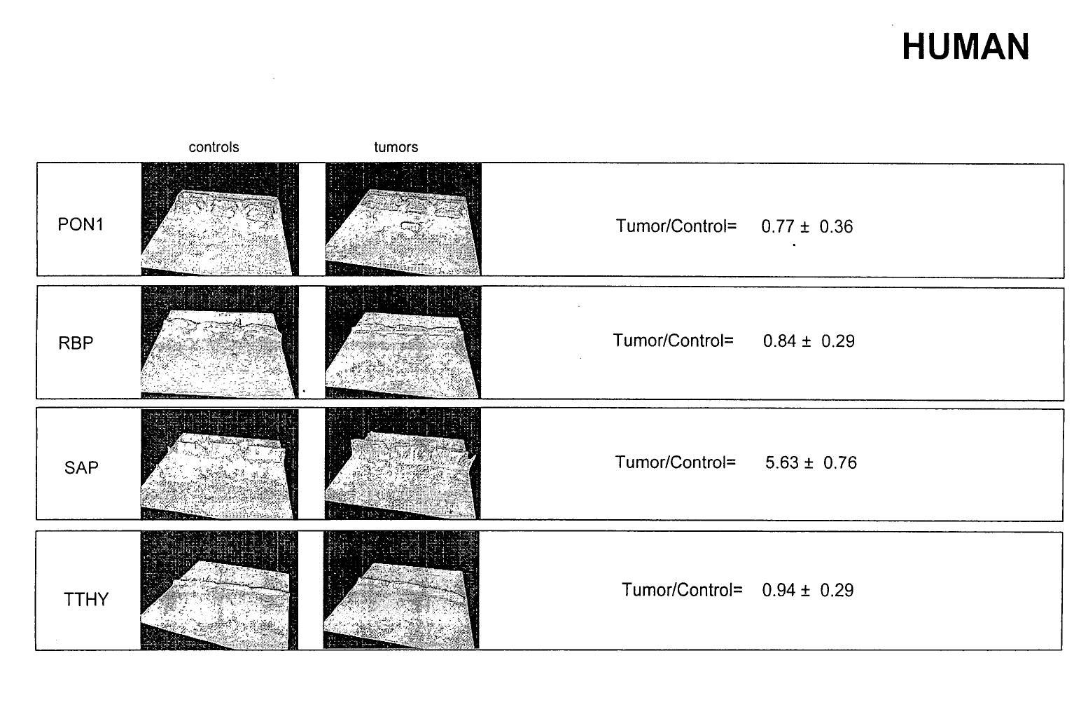

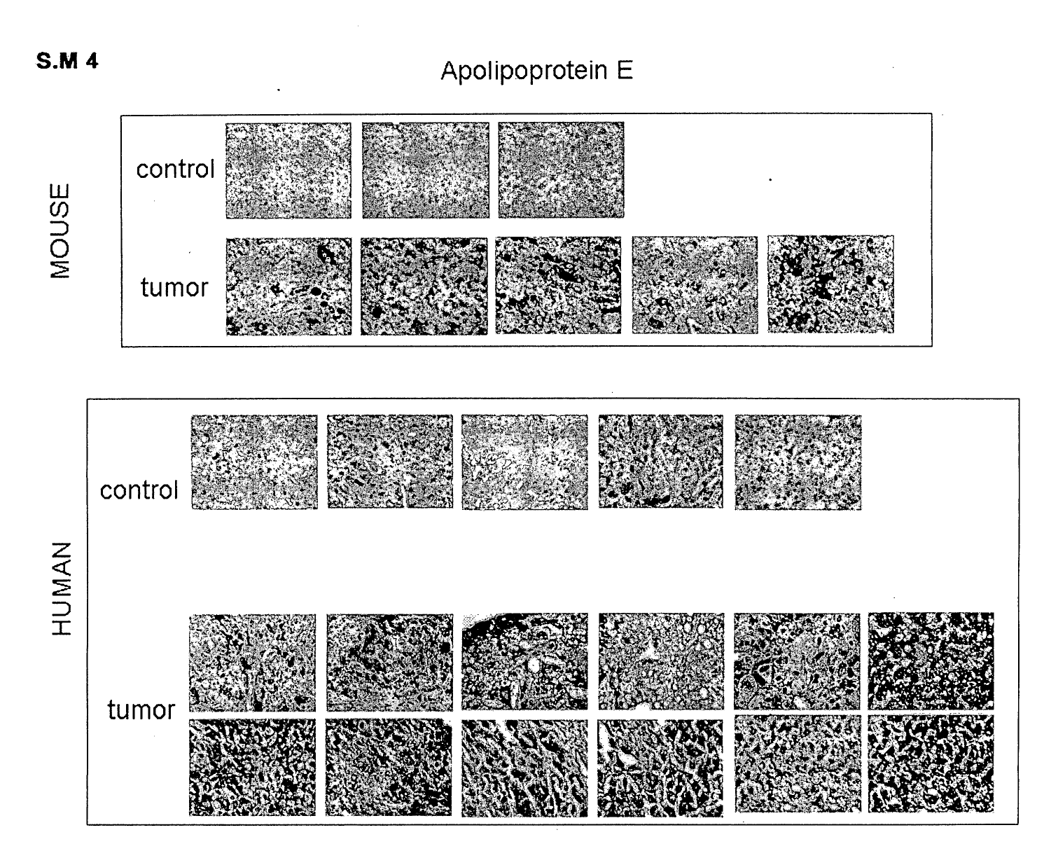

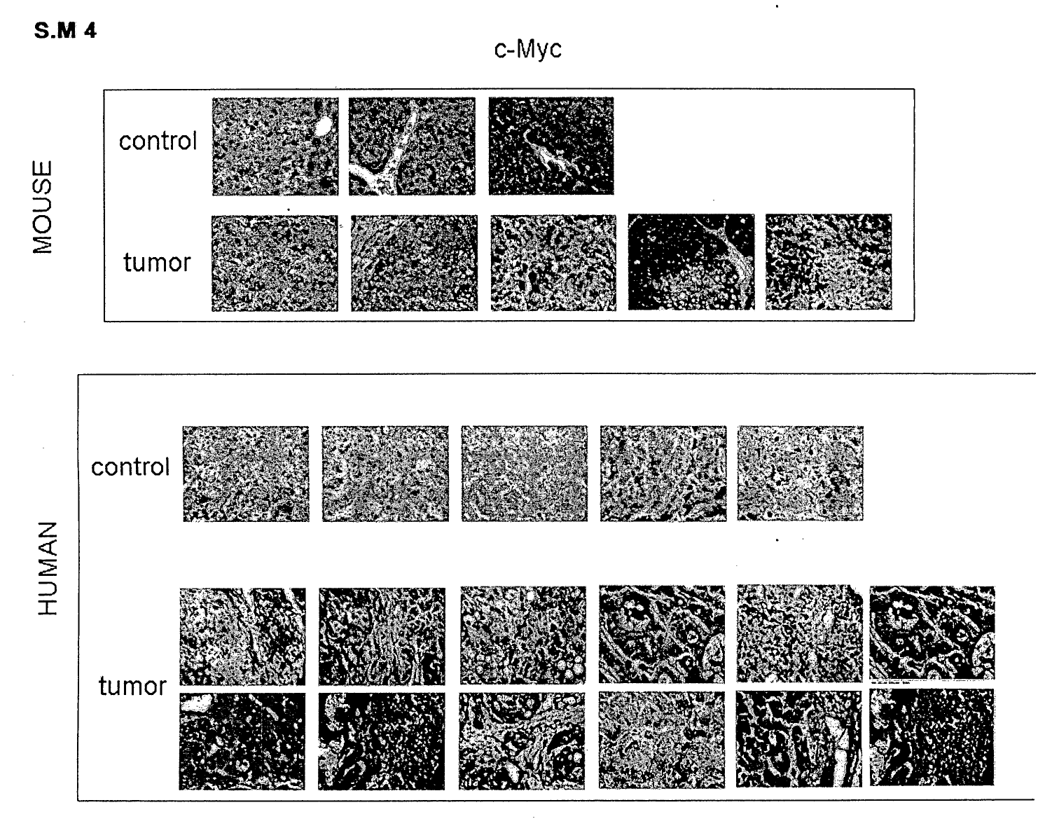

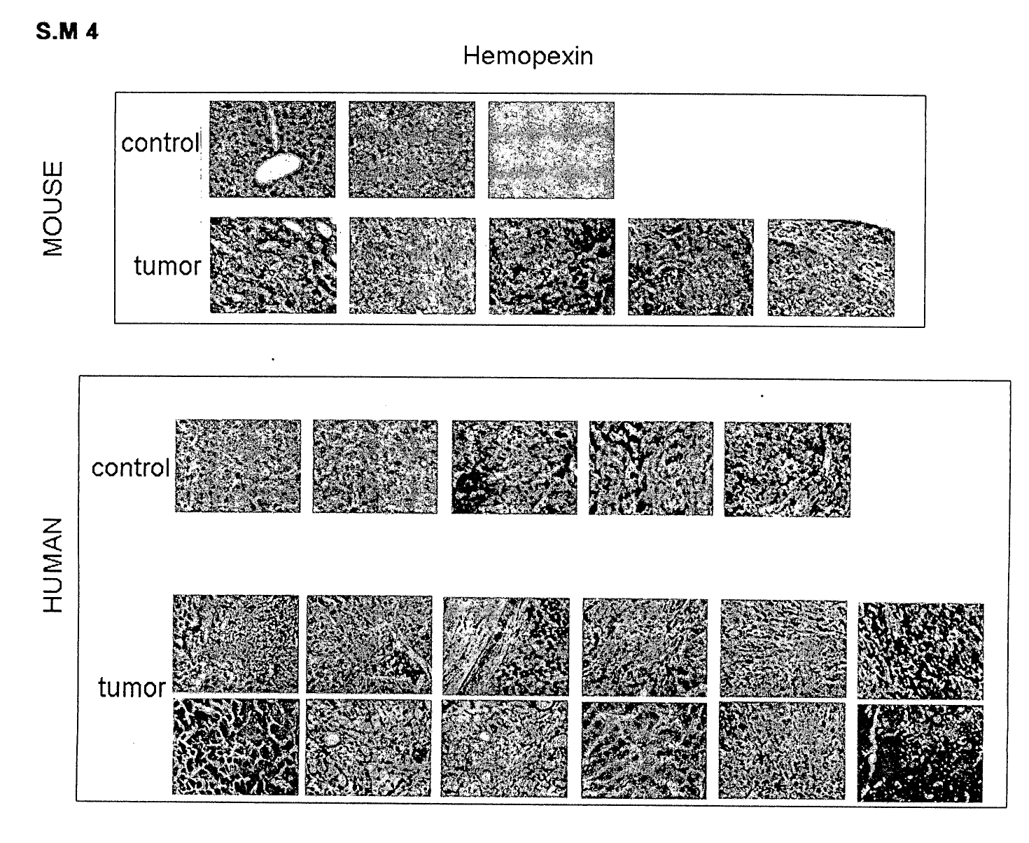

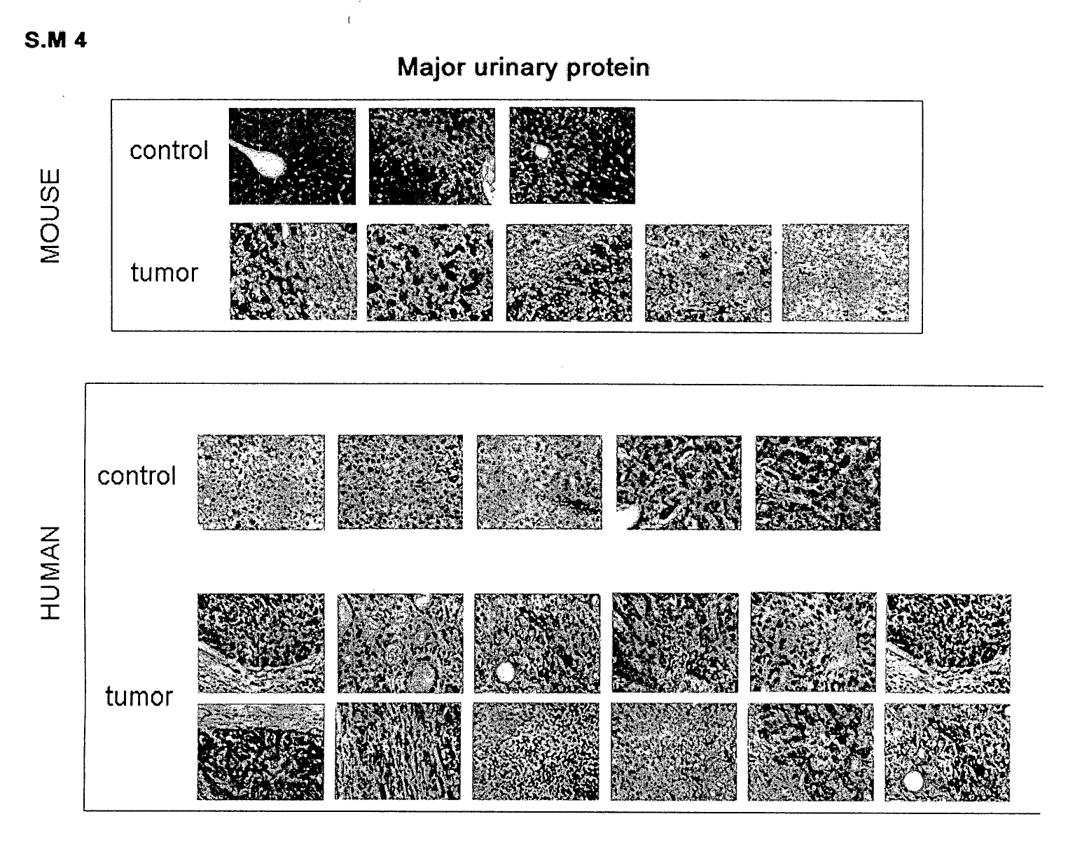

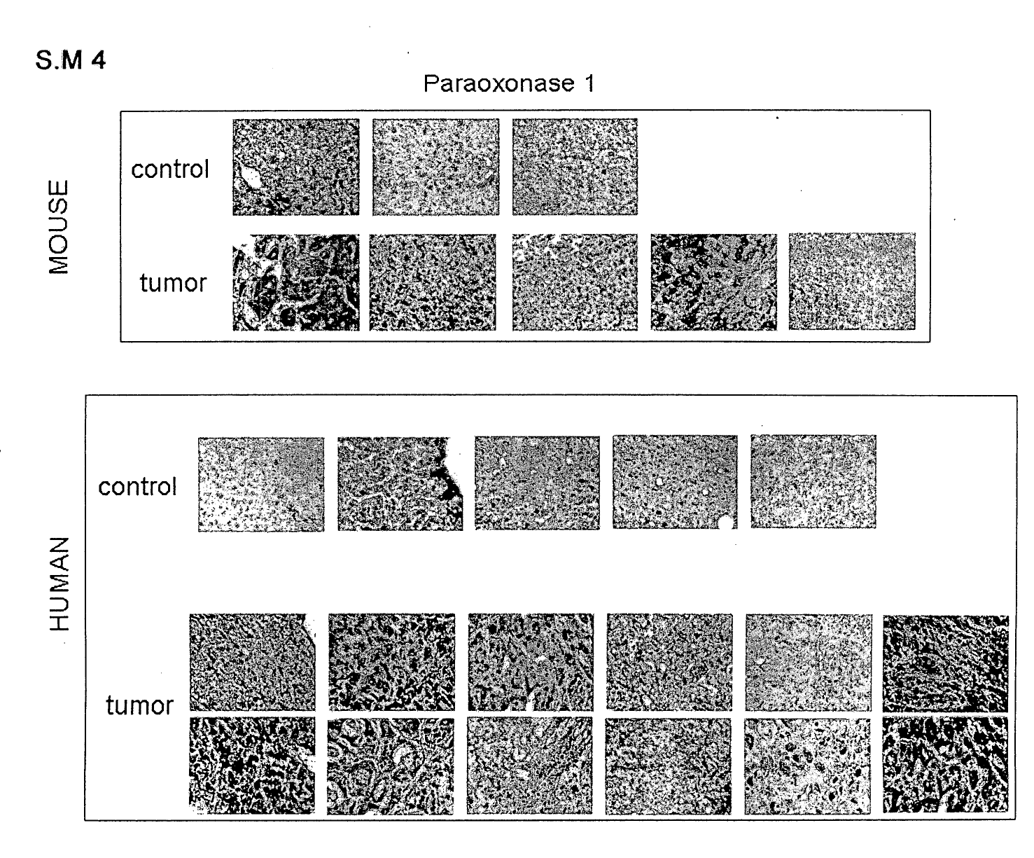

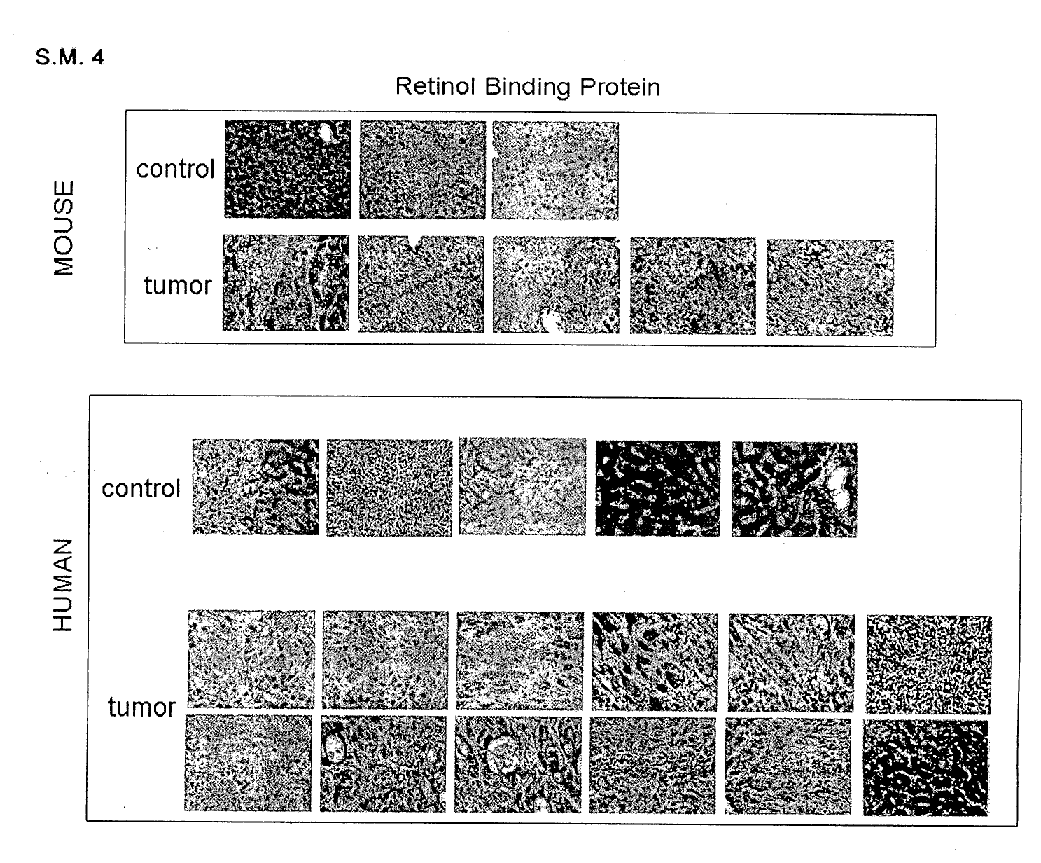

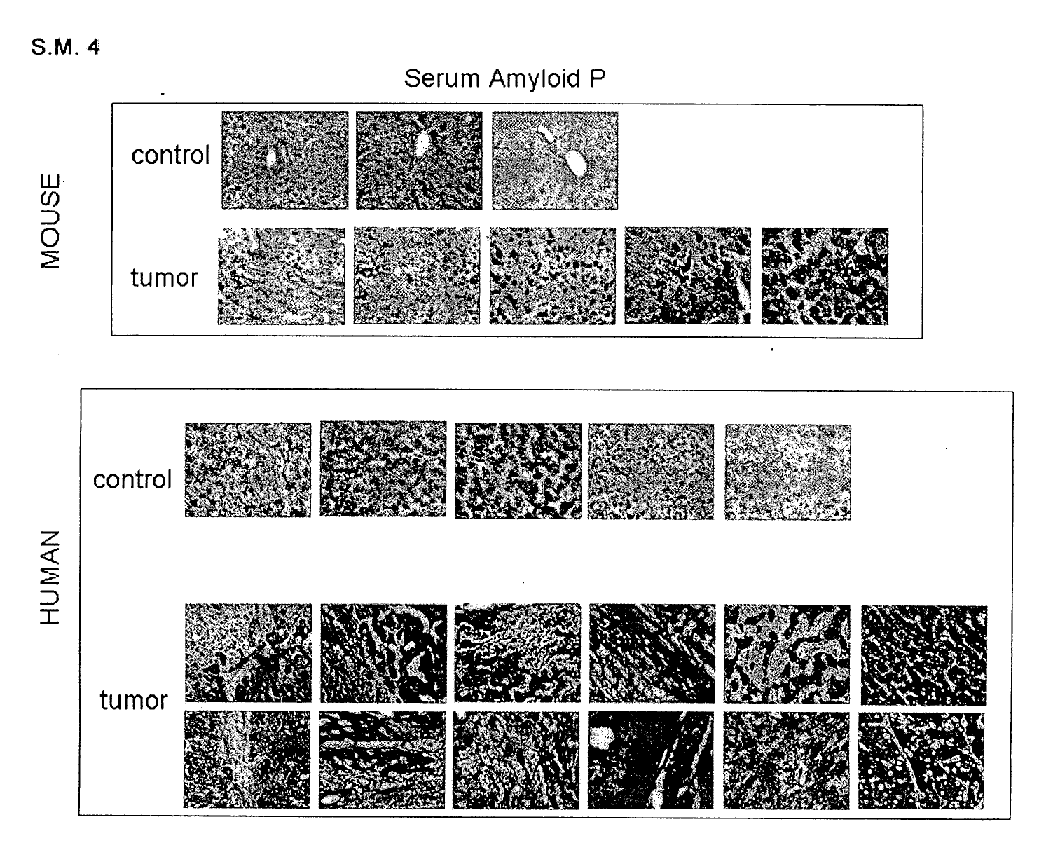

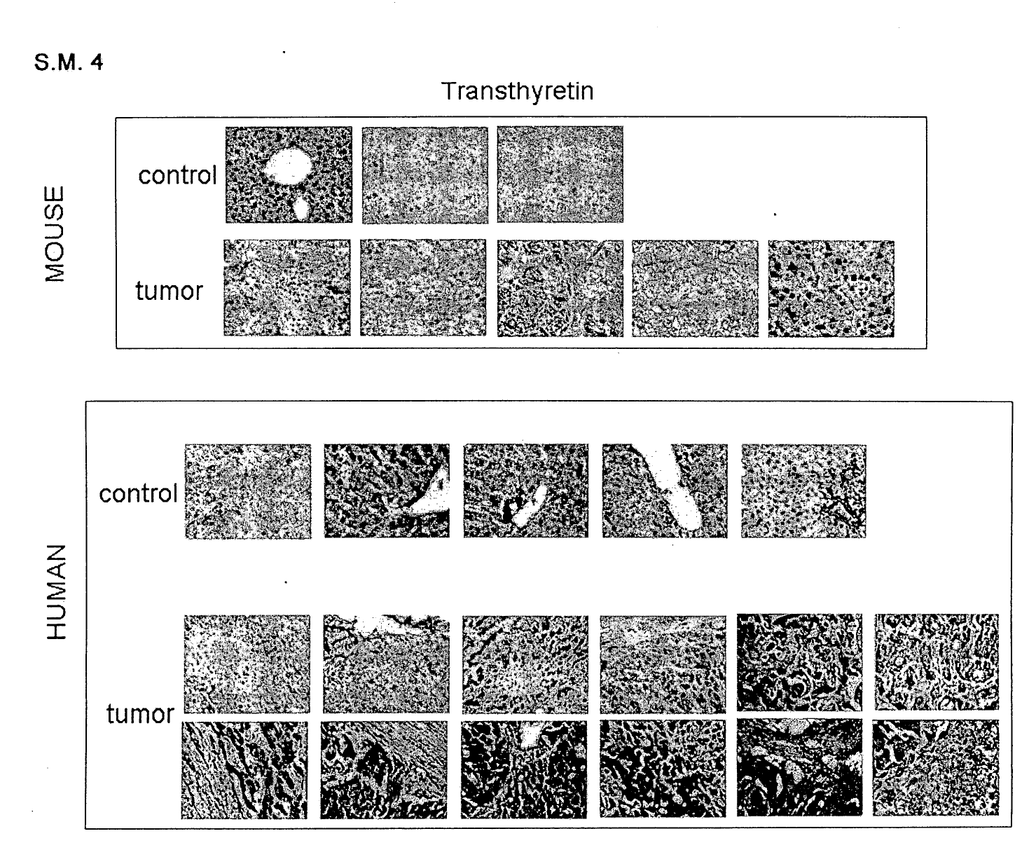

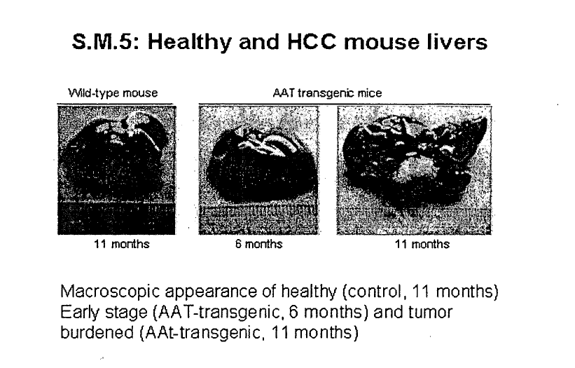

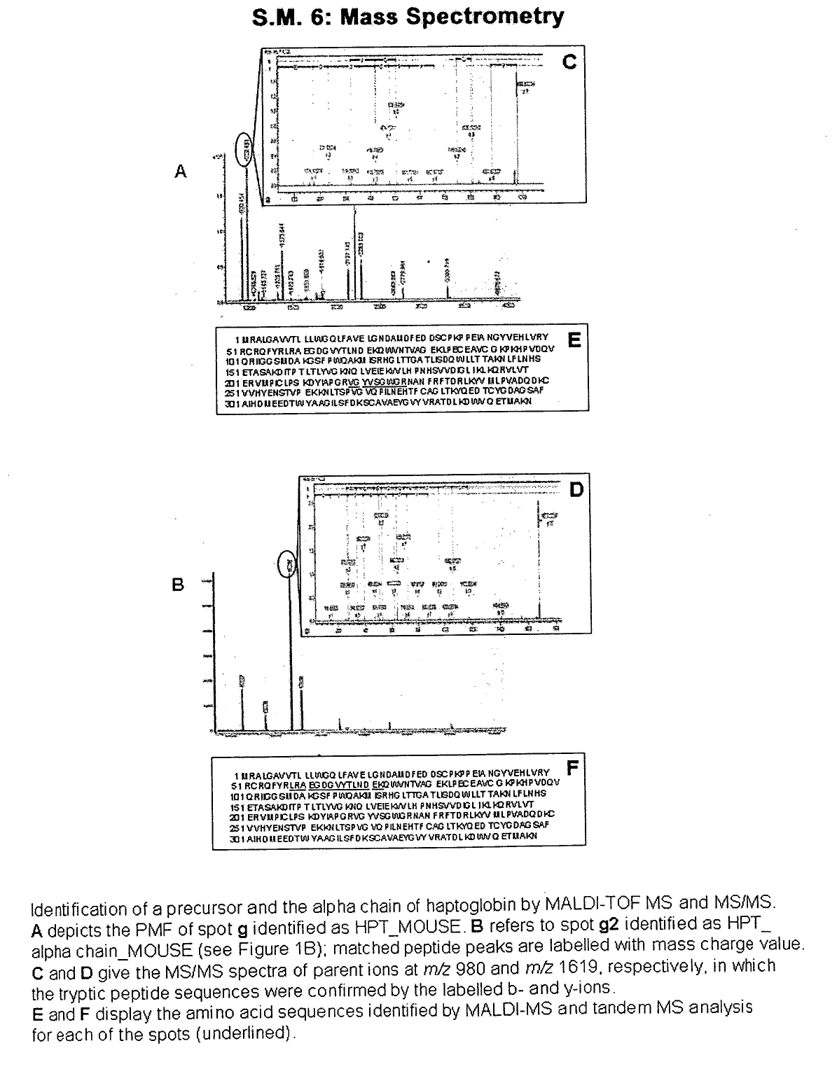

The newly identified biomarker candidates are part of different biological processes. FIG. 6 provides an overview of process, function and component associations based on beta-PubGene annotations (PubGene Inc., www.pubgene.org). Notably, in the study of Liu et al [23] cDNA clones generated from HCC libraries were screened for transcript regulation. The investigators found apolipoprotein M as well as alpha-fetoprotein to be up-regulated, whereas transcripts coding for fibrinogen, haptoglobin and hemopexin were repressed. Our study on serum proteins agree well with these findings. For instance, apolipoprotein M was up-regulated by four-fold (P<0.05) and regulation of alpha-fetoprotein and apolipoprotein M was also confirmed by WB (FIGS. 1B, 3A and 3B). Furthermore, we found hemopexin by two-fold repressed (P<0.05); again confirming earlier studies on HCC, while the precursor and the alpha-chain of haptoglobin was strongly up-regulated. Specifically, haptoglobin originates primarily from the liver and is elevated in infections, inflammations but also in malignancies [21]. Elevated levels of alphal- and alpha2- haptoglobin were reported for lung and ovarian cancer [22]. Because of their structural and functional homology with 7S immunoglobulins, alpha-haptoglobin may act as an immunosuppressant and the constitutive haptoglobin homodimeric complex may be abolished in cancer. The human heavy beta-chain of haptoglobin displays high homology with the catalytic domain of serine protease and relevant up-regulation of haptoglobin in hepatic cirrhosis and HCC-tumor cases have been reported [24]. Thus, our transgenic mouse model recapitulates such regulation as we observed a five- and two-fold up-regulation of a 40 kDa and an 8-9 kDa haptoglobin in the serum of HCC bearing mice.

Likewise, mouse serum amyloid P (SAP) was up-regulated, that is a member of the pentraxins family, but SAP is a more distant relative of the “long” pentraxins such as PTX3 (a cytokine modulated molecule) as well as several neuronal pentraxins. Notably, SAP shares 51% sequence homology with C-reactive protein (CRP), an acute phase protein and SAP and CRP are evolutionary conserved. Again, our findings with murine SAP agree well with regulations reported for human serum and tissue (see FIGS. 3A/B and 5A/B).

We also observed regulation of apolipoprotein E that functions as an acute phase-reactive protein. Indeed, recent works identified apoE as a HCC biomarker candidate [25] in brain, breast, ovarian and prostate cancers and was shown to inhibit apoptosis by altering 13-catenin distribution [26]. It is of considerable importance that due to genetic polymorphisms of the apoE genes four or more isoproteins can be separated at pH 5-6 on IEF gels [27,28]. Here we identified two fragments of the apolipoprotein E (Tables 1,2); its regulation was confirmed by WB in serum and by IHC in tumor tissue (FIGS. 3 and 5A/B). Interestingly, one band at lower Mw than expected was uniquely detected by WBs in serum of HCC bearing transgenic mice.

A further disease regulated protein is hemopexin, a serum glycoprotein that binds heme for its transport to the liver and plays a pivotal role in iron recovery. Down-regulation of this protein was already reported for patients with NSCLC while in the same patient population haptoglobin was overexpressed [29]. These suggest co-regulation of these proteins and our results agree well with finding reported for NSCLC. Specifically, we observed hemopexin to be strongly up-regulated in tumor tissue as evidenced by IHC. Unfortunately, none of the tested antibodies allowed us to detect the protein by WB.

Another highly interesting finding of our study is the disease specific regulation of afamin in serum of HCC bearing mice (afamin, α-albumin, α₁T-glycoprotein). This protein is the newest member of the albumin family comprising albumin, α-fetoprotein, and vitamin D binding protein and is predominantly expressed in liver and kidney to function as a negative acute phase protein. At least three different isoforms at pH 5.05-5.25 have been identified in plasma/serum, cerebrospinal and follicular fluids. Decreased tissue levels of afamin have been associated with hepatocellular carcinoma but until now serum levels were not examined. We identified 5 spots (see FIG. 1A), two of them being statistically significantly down-regulated as determined by the PDQuest™ software. In human HCC afamin serum levels were below the limit of detection (FIG. 3B). Since down-regulation of afamin promotes proliferation [30], our works stimulates further research to better understand afamin's role in HCC. Additionally, fibrinogen and fibrinogen degradation products were reported to be regulated in several tumor types. In a lung carcinoma model of fibrinogen-deficient mice, cancer progression was diminished in the absence of fibrinogen [31]. While, fibrin/fibrinogen deposition induced fibrinolytic activity of mainly plasmin, degradation of extracellular matrix provided a fertile ground for tumor cell invasion and metastasis. Although we did not observe plasminogen to be strongly regulated (data not shown), we found fibrinogen to be overexpressed as it was reported for pancreatic cancers [32,33]. Furthermore, recent studies [34] demonstrated plasma apoM levels to be significantly increased in HCC patients, with apoM levels being even higher in patients suffering from chronic hepatitis and liver cirrhosis. We previously reported an up-regulation of apoM in an EGF-transgenic mouse model of HCC [32] and now extend this finding to a further transgenic mouse model of HCC. Thus, apoM should be considered as a bona fide biomarker candidate for HCC. Interestingly, we observed decreased expression of glutathione peroxidase 3 in HCC mice. This oxidoreductase is an important component of the defense system that involves several enzymes such as SOD and Gpx3 [35]. Down-regulation of GPx-3 glycoprotein has been reported for prostate cancer and there appears to be an inverse association between GPX3 expression and tumor grade as reported elsewhere [36]. Recent studies on the human hepatoma cell lines HepG2 also evidenced a dramatic down-regulation of oxidoreductase enzymes [37]. Overall, mapping the serum proteome of the AAT c-Myc model enabled an identification of several disease regulated proteins that have not been reported so far in the context of HCC. Specifically, paraxonase/arylesterase 1 functions as a calcium-dependent esterase that catalyzes the hydrolysis of various aromatic carboxylic acid esters and several organophosphates. Even though paraxonase/arylesterase 1 was reported to be down-regulated in human HCC [38], our findings in murine serum, in human and mouse HCC tissues are opposite (FIG. 3). Some tumor markers of HCC, such as CEA, ALP, glypican 3 are glycosylphosphatidylinositol (GPI)-anchored proteins and receive much attention as specific marker for HCC. Indeed, phosphatidylinositol-glycan-specific phospholipase D (PHLD) plays a role in degrading cell-surface proteins of the membrane through a glycosyl-phosphatidylinositol anchor (GPI) [39]. We propose PHLD as specific candidate biomarker. PHLD may reshuffle phosphatidic acid (PA) in the outer leaflet of the lipid bilayer. It is known that the addition of the PA to various mammalian cells results in Ca²⁺ mobilization, followed by a decrease of cellular levels of cAMP, an increase in DNA synthesis as well as proto-oncogene activation possibly as a result of an increase of inositol phospholipid hydrolysis [39].

We also found polymeric immunoglobulin receptor to be exclusively expressed in serum samples of HCC bearing mice and confirmed this finding for HCC in patients. This protein is located on the basolateral surface of mucosal cells to bind dimeric IgA produced by B cells for cooperation with T cells in the lamina propria. Normally, the secretory immunoglobulin A (IgA) prevents pathogen adherence at mucosal surfaces to prevent infection. Importantly, PIgR is known to be down-regulated in nasopharyngeal carcinoma [40]. Here we reported this protein to be uniquely expressed in HCC; a regulatory c-Myc sequence was identified in the promoter for this gene PigR may be classified as a target of c-Myc [41].

Unlike tissue levels, we found major urinary protein (MUP) to be down-regulated in the serum of tumor bearing mice. Unfortunately, we were unable to separate and hence identify the specific isoforms that might have been regulated even though recent investigators found MUP11&8 to be strongly down-regulated in HCC in a SOD deficient mouse model [42]. In our IHC study with human HCC a MUP antibody was used that cross-reacts with an orthologe epitope but it remains to be determined whether human MUP is truly expressed in liver. So far, only a pseudo-gene was reported [43].

Finally, the serum proteome of another genetic model of HCC was reported where over-expression of the epidermal growth factor (EGF) resulted in liver cancer [32]. We compared the findings amongst the two different transgenic mouse models of HCC. Indeed, several proteins, i.e., alpha-fetoprotein, apolipoproteins E and M, fibrinogen-beta, glutathione peroxidase 3, major urinary protein and serum amyloid P were commonly regulated while expression of hemopexin, haptoglobin, phosphatidylinositol-glycan-specific phospholipase D, polymeric immunoglobulin receptor, paraoxonasel, retinol binding protein, serine protease inhibitor A3K and transthyretin was uniquely regulated in the AAT c-Myc mouse model of HCC. Likewise, we found alpha-2-macroglobulin to be exclusively regulated in the EGF-HCC model, therefore evidencing biomarkers to be specifically linked to various pathologies induced by different mitogens, i.e exaggerated EGF tyrosine kinase or induced myc activities.

In conclusion, we report novel serum and tissue biomarkers of HCC worthy for their in depth clinical evaluation.

### Figure Legends

### FIG. 1A “Examples of Down-Regulated Proteins”

Representative control 2-D gel

### 1B “Examples of Up-Regulated Proteins”

Representative tumor 2-D gel and differentially expressed spots at pH 4-7. Protein spots are visualized by Coomassie blue staining.

### FIG. 2 “2-DE and Western Immunoblotting”

Regulated serum proteins were validated by Western immunoblotting (WB). Serum proteins (50 ug) from 3 controls and 3 AAT-c-myc transgenic mice were separated by 8%, 12% and15% SDS-PAGE. Total protein extracts from HepG2 and Hela total cell extracts were loaded as positive control. The loading was controlled by alpha-tubulin. Data from 2-DE are shown in Table 1 and Supplementary Material 2. Histograms display statistically significant fold changes (T/C±RSD) determined from 2-D gels at pH 4-7 (“single squaring” [symbol] ) and 3-10 (“double squaring” [symbol] ).

### FIG. 3

### A. “Protein Expression at Different Stages of Disease”

WBs of n=3 healthy, 5.5, 6, 6.5 months old mice and HCC bearing transgenic mice are compared.

### B. “Western Immunoblotting of Regulated Serum Proteins in HCC Patients”

Serum of n=11 healthy and n=20 HCC patients (data in Supplementary Material 1) was analyzed with the QDots™ kit (Invitrogen) in case of Prestige Antibodies®. HepG2 and Hela total cell extracts (50 ug) were loaded as positive controls.

### FIG. 4 “Histograms”

Quantitation of WB bands of human serum samples by QuantityOne® software. We show the protein regulation (fold-change ±RSD) in case of viral infection (A), C2 (B) or in all other cases (C).

### FIG. 5 “Immunohistochemistry”

### A and B.

Immunohistochemistry of murine and human liver tissues (Images depict results at 10× and 40×).

### FIG. 6 “GO Classification of Regulated Proteins in HCC”

The differentially expressed proteins were classified according to Gene Ontology by PubGene (component associations, function associations and biological process, www.pubgene.org).

### REFERENCES

1. El-Serag H B, Rudolph K L. Hepatocellular carcinoma: Epidemiology and Molecular Carcinogenesis. Rev Basic Clin Gastroenterol 2007;132:2557-76.
2. American Cancer Society's website [http://www.cancer.org/docroot/CRI/CRI_{—}2_{—}3x.asp?dt=25].
3. Hoffmann K, Schmidt J. [New surgical approaches in the treatment of hepatocellular carcinoma]. Z Gastroenterol. 2009;47:61-7.
4. Zinkin N T, Grail F, Bhaskar K, Otu H H, Spentzos D, Kalmowitz B, et al. Serum proteomics and biomarkers in hepatocellular carcinoma and chronic liver disease. Clin Cancer Res. 2008;14:470-7.
5. Harino Y, Fujii M, Imura S, Morine Y, Ikemoto T, Soejima Y, et al. The role of desgamma-carboxyprothrombin expression in hepatocellular carcinoma. Hepatogastroenterology. 2008;55:1385-9.
6. Marrero J A, Feng Z, Wang Y, Nguyen M H, Befeler A S, Roberts L R et al. α-Fetoprotein, Des-γ Carboxyprothrombin, and Lectin-Bound α-Fetoprotein in Early Hepatocellular Carcinoma. Gastroenterology 2009; 137:110-118
7. Srivastava S. Cancer Biomarker Discovery and Development in Gastrointestinal Cancers: Early Detection Research Network—A Collaborative Approach. Gastrointestinal Cancer Research. 2007; S60-S63
8. Ucar E, Semerci E, Ustun H, Yetim T, Huzmeli C, Gullu M. Prognostic value of preoperative CEA, CA 19-9, CA 72-4, and AFP levels in gastric cancer. Adv Ther. 2008;10:1075-84.
9. Sherman M. Alphafetoprotein: an obituary. J Hepatol 2001;34:603-5.
10. Shachaf C M, Gentles A J, Elchuri S, Sahoo D, Soen Y, Sharpe O, et al. Genomic and proteomic analysis reveals a threshold level of MYC required for tumor maintenance. Cancer Res. 2008;13:5132-42.
11. Dalemans W, Perraud F, Le Meur M, Gerlinger P, Courtney M, Pavirani A. Heterologous protein expression by transimmortalized differentiated liver cell lines derived from transgenic mice (hepatomas/alpha 1 antitrypsin/ONC mouse). Biologicals. 1990;18:191-8.
12. Ritorto M S, Borlak J. A simple and reliable protocol for mouse serum proteome profiling studies by use of two-dimensional electrophoresis and MALDI TOF/TOF mass spectrometry. Proteome Sci 2008;6:25.
13. Marengo E, Robotti E, Bobba M, Multivariate statistical tools for the evaluation of proteomics 2D-maps: recent achievements and applications. Current Proteomics 2007;4:53-66.
14. Fuxius S, Eravci M, Broedel O, Weist S, Mansmann U, Eravci S, Baumgartner A, Technical strategies to reduce the amount of “false significant” results in quantitative proteomics. Proteomics 2008;9:1780-4.
15. Garaguso I, Borlak J. Matrix layer sample preparation: an improved MALDI-MS peptide analysis method for proteomic studies. Proteomics 2008;13:2583-95.
16. Molloy M P, Brzezinski E E, Hang J, McDowell M T, VanBogelen R A. Overcoming technical variation and biological variation in quantitative proteomics. Proteomics. 2003;10:1912-9.
17. Asirvatham V S, Watson B S, Sumner L W. Analytical and biological variances associated with proteomic studies of Medicago truncatula by two-dimensional polyacrylamide gel electrophoresis. Proteomics 2002;8:960-8.
18. Paradis V, Bedossa P. In the new area of noninvasive markers of hepatocellular carcinoma. J Hepatol. 2007;46:9-11.
19. Zhang Z, Bast R. C, Yu Y, Li J, Sokoll L J, Rai A J, et al. Three biomarkers identified from serum proteomic analysis for the detection of early stage ovarian cancer. Cancer Res. 2004;64:5882-5890.
20. Meistermann H, Norris J L, Aerni H R, Cornett D S, Friedlein A, Erskine A R, et al. Biomarker discovery by imaging mass spectrometry: transthyretin is a biomarker for gentamicin-induced nephrotoxicity in rat. Mol Cell Proteomics. 2006;10:1876-86.
21. Zhao C, Annamalai L, Guo C, Kothandaraman N, Koh S C, Zhang H, et al. Circulating haptoglobin is an independent prognostic factor in the sera of patients with epithelial ovarian cancer. Neoplasia 2007;1:1-7.
22. Ye B, Cramer D W, Skates S J, Gygi S P, Pratomo V, Fu L, Horick N K, et al. Haptoglobin-alpha subunit as potential serum biomarker in ovarian cancer: identification and characterization using proteomic profiling and mass spectrometry. Clin Cancer Res 2003;8:2904-11.
23. Liu Y, Zhu X, Zhu J, Liao S, Tang Q, Liu K, et al. Identification of differential expression of genes in hepatocellular carcinoma by suppression subtractive hybridization combined cDNA microarray. Oncol Rep. 2007;18:943-51.
24. Gravel P, Walzer C, Aubry C, Balant LP, Yersin B, Hochstrasser D F, et al. New alterations of serum glycoproteins in alcoholic and cirrhotic patients revealed by high resolution two-dimensional gel electrophoresis. Biochem Biophys Res Commun. 1996 ;220:78-85.
25. Yokoyama Y, Kuramitsu Y, Takashima M, lizuka N, Terai S, Oka M, Nakamura K, Okita K, Sakaida I. Protein level of apolipoprotein E increased in human hepatocellular carcinoma. Int J Oncol 2006;28:625-31.
26. Caruso A, Motolese M, lacovelli L, Caraci F, Copani A, Nicoletti F, Terstappen GC, Gaviraghi G, Caricasole A. Inhibition of the canonical Wnt signaling pathway by apolipoprotein E4 in PC12 cells. J Neurochem 2006;98:364-71.
27. Reardon C A, Driscoll D M, Davis R A, Borchardt R A, Getz G S. The charge polymorphism of rat apoprotein E. J Biol Chem 1986;261:4638-45.
28. Kahri J, Soro-Paavonen A, Ehnholm C, Taskinen MR. ApoE polymorphism is associated with C-reactive protein in low-HDL family members and in normolipidemic subjects. Mediators lnflamm 2006;3:12587.
29. Dowling P, O'Driscoll L, Meleady P, Henry M, Roy S, Ballot J, Moriarty M, et al. 2-D difference gel electrophoresis of the lung squamous cell carcinoma versus normal sera demonstrates consistent alterations in the levels of ten specific proteins. Electrophoresis 2007;28:4302-10.
30. Wu GX, Lin YM, Zhou TH, Gao H, Pei G. Significant down-regulation of 60 -albumin in human hepatoma and its implication. Cancer Lett 2000;160:229-36.
31. Pollanen J, Stephens R W, Vaheri A. Direct plasminogen activation at the surface of normal and malignant cells. Adv Cancer Res 1991;57:273-328.
32. Gazzana G, Borlak J. Mapping of the serum proteome of hepatocellular carcinoma induced by targeted overexpression of epidermal growth factor to liver cells of transgenic mice. J Proteome Res 2008; 7:928-37.
33. Bloomston M, Zhou J X, Rosemurgy A S, Frankel W, Muro-Cacho C A, Yeatman T J. Fibrinogen gamma overexpression in pancreatic cancer identified by large-scale proteomic analysis of serum samples. Cancer Res 2006;66:2592-9.
34. Jiang J, Zhang X, Wu C, Qin X, Luo G, Deng H, et al. Increased plasma apoM levels in the patients suffered from hepatocellular carcinoma and other chronic liver diseases. Lipids Health Dis 2008;7:25.
35. Yang F, Yan S, He Y, Wang F, Song S, Guo Y, et al. Expression of hepatitis B virus proteins in transgenic mice alters lipid metabolism and induces oxidative stress in the liver. J Hepatol 2008;48:12-9.
36. Zhuo P, Diamond A M. Molecular mechanisms by which selenoproteins affect cancer risk and progression. Biochim Biophys Acta. 2009;13.
37. Ngoka L C. Dramatic down-regulation of oxidoreductases in human hepatocellular carcinoma hepG2 cells: proteomics and gene ontology unveiling new frontiers in cancer enzymology. Proteome Sci. 2008;6:29.
38. Yang M H, Tyan Y C, Jong S B, Huang Y F, Liao P C, Wang M C. Identification of human hepatocellular carcinoma-related proteins by proteomic approaches. Anal Bioanal Chem 2007;388:637-43.
39. Low M G, Prasad A R. A phospholipase D specific for the phosphatidylinositol anchor of cell-surface proteins is abundant in plasma. Proc Nati Acad Sci U S A 1988;85:980-4.
40. Chang Y, Lee T C, Li J C, Lai T L, Chua H H, Chen C L, et al. Differential expression of osteoblast-specific factor 2 and polymeric immunoglobulin receptor genes in nasopharyngeal carcinoma. Head Neck 2005;27:873-82.
41. Bruno M E C, West R B, Schneeman T A, Bresnick E H, Kaetzel C S. Upstream stimulatory factor but not c-Myc enhances transcription of the human polymeric immunoglobulin receptor gene. Mol Immun 2004;40:695-708.
42. Elchuri S, Naeemuddin M, Sharpe O, Robinson W H, Huang T T. Identification of biomarkers associated with the development of hepatocellular carcinoma in CuZn superoxide dismutase deficient mice. Proteomics 2007;7:2121-9.
43. Chamero P, Marton T F, Logan D W, Flanagan K, Cruz J R, Saghatelian A, Cravatt BF, et al. Identification of protein pheromones that promote aggressive behaviour. Nature. 2007;450:899-902.

### Table Legends

TABLE 1
List of the 17 differentially expressed proteins in the serum of HCC bearing mice.
Mass spectrometry values were chosen according the best results. In “Number of
peptides” section, ox (= oxidation) is the number of oxidated peptides given by
MASCOT. Fold change (FC, T/C) was determined from gels at the pH 4-7 and 3-10,
respectively. Coefficients of variation (CV) are given in brackets. T designates serum
proteins found in HCC bearing mice only.

TABLE 2
Statistical validation: Statistical data were collected from the PDQuest ™ data
analysis. Student-t- test, Mann U-Whitney test for univariate and PLS for multivariate
analyses were computed at a statistical significance of P < .05 and P < .01. The best
results are reported (a).
MS validation: MALDI TOF/TOF tandem MS of selected matched peptides to the
sequence from PMF spectra (b).
Immunovalidation: Western blots were quantified (T/C ± RSD) by the Quantity One ®
software, after local subtraction of background and normalization with α-tubulin (c).

The characteristics of the invention being disclosed in the preceding description, the subsequent drawings and claims can be of importance both singularly and in arbitrary combination for the implementation of the invention in its different embodiments. The foregoing description of preferred embodiments of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications to thereby enable others skilled in the art to best utilize the invention in various embodiments and with various modifications as are suited to the particular use contemplated invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

TABLE 1
Regulated proteins in AAT c-Myc mice

| | | | Swiss-Prot | | | | | | | | |
|---|---|---|---|---|---|---|---|---|---|---|---|
| | Swiss-Prot | | accession | Theoretical | | Number of | Sequence | 4-7 pH | | 3-10 pH | |
| FIG. 1 | Entry | Protein Name | number | Mw/pI | Score | peptides | coverage | FC (CV %) | | FC (CV %) | |
| Alpha-1- | A1AT | Alpha-1- | P01009 | 46.9/5.37 | 219 | 24 + 1ox | 62 | T | (11) | T | (47) |
| antitrypsin 1-1 | HUMAN* | antitrypsin 1-1 | | | | | | | | | |
| 1 | AFAM | Afamin | O89020 | 71.5/5.54 | 148 | 17 + 3ox | 33 | 0.7 | (20) | 0 | (55) |
| | MOUSE | | | | | | | | | | |
| AFP | AFP | Alpha-fetoprotein | P02772 | 67.3/5.54 | 203 | 16 | 38 | 1.8 | (74) | 1.8 | (57) |
| | MOUSE | | | | | | | | | | |
| 13 | ANT3 | Antithrombin 3 | P32261 | 52.5/6.1 | 183 | 21 + 3ox | 46 | 2.6 | (32) | / | |
| | MOUSE | | | | | | | | | | |
| 9 | APOE | Apolipoprotein E | P08226 | 35.9/5.56 | 259 | 24 + 1ox | 53 | 2.2 | (26) | 2.2 | (66) |
| | MOUSE | | | | | | | | | | |
| 9.1 | APOE | Apolipoprotein E | P08226 | 35.9/5.56 | 74 | 7 + 2ox | 19 | T | (9.7) | T | (26) |
| | MOUSE | | | | | | | | | | |
| 9.2 | APOE | Apolipoprotein E | P08226 | 35.9/5.56 | 83 | 10 + 1ox | 26 | T | (138) | T | (16) |
| | MOUSE | | | | | | | | | | |
| 8 | APOM | Apolipoprotein M | Q9Z1R3 | 21.6/6.8 | 105 | 8 + 1ox | 36 | 4.2 | (20) | T | (65) |
| | MOUSE | | | | | | | | | | |
| 11 | FIBB | Fibrinogen beta- | Q8K0E8 | 55.4/6.68 | 155 | 17 + 1ox | 40 | T | (45) | / | |
| | MOUSE | chain | | | | | | | | | |
| 2 | GPX3 | Glutathione | P46412 | 25.6/8.26 | 98 | 12 | 45 | 0.5 | (19) | 0.4 | (74) |
| | MOUSE | peroxidase 3 | | | | | | | | | |
| 3 | HEMO | Hemopexin | Q91X72 | 52/7.92 | 260 | 23 | 43 | 0.5 | (35) | 0.3 | (72) |
| | MOUSE | | | | | | | | | | |
| 7 | HPT | Haptoglobin | Q61646 | 39.2/5.88 | 159 | 11 + 2ox | 41 | 5.5 | (27) | T | (38) |
| | MOUSE | | | | | | | | | | |
| 7.1 | HPTαchain | Haptoglobin | Q61646 | 39.2/5.88 | 75 | 6 | 12 | 2.2 | (28) | 2.6 | (38) |
| | MOUSE | | | | | | | | | | |
| 4 | MUP2-6-8-1 | Major urinary protein | P11589 | 20.9/5.04 | 184 | 13 | 74 | 0.07 | (53) | / | |
| | MOUSE | | | | | | | | | | |
| 15 | PHLD | Phosphatidylinositol- | O70362 | 93.8/6.65 | 151 | 20 | 23 | 5.2 | (29) | / | |
| | MOUSE | glycan-specific | | | | | | | | | |
| | | phospholipase D | | | | | | | | | |
| 12 | PIGR | Polimeric | O70570 | 86.2/5.26 | 162 | 15 | 23 | T | (22) | / | |
| | MOUSE | immunoglobulin | | | | | | | | | |
| | | receptor | | | | | | | | | |
| 14 | PON1 | Paraoxonase 1 | P52430 | 39.6/5.07 | 137 | 11 | 39 | 1.9 | (25) | 2 | (49) |
| | MOUSE | | | | | | | | | | |
| 6 | RETBP | Retinol binding | Q00724 | 23.5/5.69 | 154 | 12 | 61 | 2.2 | (32) | 7 | (30) |
| | MOUSE | protein | | | | | | | | | |
| 16 | SAMP | Serum amyloid P- | P12246 | 26.4/6 | 99 | 8 | 34 | 2.4 | (73) | / | |
| | MOUSE | component | | | | | | | | | |
| 5 | SPA3K | Serine protease | P07759 | 47.02/5.05 | 103 | 12 + 1ox | 50 | 0.3 | (44) | / | |
| | MOUSE | inhibitor A3K | | | | | | | | | |
| 10.1 | TTHY | Transthyretin | P07309 | 15.9/5.77 | 133 | 8 | 67 | 1.6 | (25) | 1.9 | (29) |
| | MOUSE | | | | | | | | | | |
| 10 | TTHY | Transthyretin | P07309 | 15.9/5.77 | 87 | 7 | 51 | 1.1 | (30) | 2.6 | (35) |
| | MOUSE | | | | | | | | | | |

*human A1AT as part of the gene construct to produce transgenic mice (see Materials and Methods)

TABLE 2
Protein identification by MALDI TOF and immuno

[symbol]

| | | | | | Mass spectrometer validation b) | | | Immuno validation c) | | |
|---|---|---|---|---|---|---|---|---|---|---|
| | | | | | MS/MS | | | | Semi- | |
| Identification | | Statistical | | | peptide | | | | Quanti- | Immuno- |
| | Protein | validation a) | | | mass | PFF | | Western | tative | histo |
| Spot | Name | t-test | Mann | PLS | observed | Score | Sequence (AA) | Blot | (T/C) | chemistry |
| Alpha-1- | A1AT | P < .01 | P < .01 | P < .01 | / | / | / | | | |
| antitryp | HUMAN | | | | | | | | | |
| sin 1-1 | | | | | | | | | | |
| 1 | AFAM | P < .05 | P > .05 | P < .01 | / | / | / | down | 0.21 [symbol] (h) | |
| | MOUSE | | | | | | | (human) | | |
| AFP | AFP | P < .05 | P < .05 | P > .05 | / | / | / | | 1.81 [symbol] (m) | |
| | MOUSE | | | | | | | | | |
| 13 | ANT3 | P < .05 | P < .05 | P > .05 | 1359.78 | 59 | DIPVNPLCIYR | | | |
| | Mouse | | | | | | (47-57) | | | |
| | | | | | 1700.94 | 63 | LQPLDFKENPEQSR | | | |
| | | | | | | | (203-216) | | | |
| 9 | APOE | P < .05 | P < .05 | P > .05 | 1075.58 | 59 | LQAEIFQAR | up | 2.29 [symbol] (m) | |
| | MOUSE | | | | | | (262-270) | (mouse/ | 1.95 [symbol] (h) | |
| | | | | | 1599.78 | 87 | ELEEQLGPVAEETR | human) | | |
| | | | | | | | (87-100) | | | |
| | | | | | 968.52 | 67 | LGPLVEQGR | | | |
| | | | | | | | (191-199) | | | |
| | | | | | 1599.77 | 105 | ELEEQLGPVAEETR | | | up |
| | | | | | | | (87-100) | | | |
| 9.1 | APOE | P < 05 | P < 01 | P > 05 | 1759.84 | 56 | NEVHMLGQSTEEIR | only T | Only T(m) | |
| | MOUSE | | | | | (ox) | (130-144) | (mouse) | | |
| 9.2 | APOE | P < 05 | P < 01 | P > 05 | 1599.77 | 44 | ELEEQLGPVAEETR | | | |
| | MOUSE | | | | | | (87-100) | | | |
| 8 | APOM | P < 05 | P < 01 | P < 05 | 938.42 | 27 | ETGQGYQR | up | 2.20 [symbol] (m) | |
| | MOUSE | | | | | | (138-145) | (human | 98 [symbol] (h) | |
| 11 | FIBB | P < -5 | P < 01 | P > 05 | / | / | / | | | |
| | MOUSE | | | | | | | | | |
| 2 | GPX3 | P < 05 | P < 01 | P < 01 | 1955.01 | 41 | YVRPGGFVPNFQLFEK | | | |
| | MOUSE | | | | | | (121-137) | | | |
| 3 | HEMO | P < 01 | P < 01 | P < 01 | 1928.11 | 65 | FNPVTGEVPPRYPLDAR | | | |
| | | | | | | | (208-224) | | | |
| | MOUSE | | | | 2448.07 | 151 | ELGSPPGISLETIDAAF | | | |
| | | | | | | | SCPGSSR(346-369) | | | |
| | | | | | 1727.77 | 64 | GECQSEGVLFFQGNR | | | |
| | | | | | | | (151-165) | | | |
| | | | | | 2472.15 | 90 | LFQEEFPGIPYPPDAAV | | | |
| | | | | | | | ECGR(130-150) | | | |
| | | | | | 1516.7 | 45 | GATYAFTGSHYWR | | | |
| | | | | | | | (270-282) | | | |
| | | | | | 1504.79 | 28 | WKNPITSVDAAFR | | | |
| | | | | | | | (90-120) | | | |
| | | | | | 1212.64 | 47 | FNPVTGEVPPR | | | |
| | | | | | | | (208-218) | | | |
| | | | | | 1100.47 | 41 | DYFVSCPGR | | | |
| | | | | | | | (225-233) | | | |
| | | | | | 1855.85 | 66 | GECQSEGVLFFQGNRK | | | |
| | | | | | | | (151-166) | | | |
| 7 | HPT | P < 01 | P < 01 | P < 01 | 1320.74 | 33 | DITPTLTLYVGK | | | |
| | | | | | | | (157-168) | | | |
| | MOUSE | | | | 1373.61 | 65 | SCAVAEYGVYVR | | | |
| | | | | | | | (321-332) | | | |
| | | | | | 1832.93 | 68 | VMPICLPSKDYIAPGR | | | |
| | | | | | | | (203-218) | | | |
| | | | | | 980.53 | 34 | VGYVSGWGR | | | |
| | | | | | | | (219-227) | | | |
| | | | | | 920.4 | 56 | GSFPWQAK | | | |
| | | | | | | | (112-119) | | | |
| 7.1 | HPT | P < 05 | P < 05 | P < 01 | 1679.85 | 63 | LRAEGDGVYTLNDEK | | | |
| | | | | | | | (58-72) | | | |
| | MOUSE | | | | 1387.68 | 58 | LPECEAVCGKPK | | | |
| | | | | | | | (83-94) | | | |
| 4 | MUP | P < 01 | P < 01 | P > 05 | / | / | / | down | <0.01(m) | up |
| | MOUSE | | | | | | | (mouse/ | | |
| | | | | | | | | human) | | |
| 15 | PHLD | P < 05 | P < 05 | P > .05 | / | / | / | up | 8.29 [symbol] (m) | |
| | MOUSE | | | | | | | (mouse/ | 2.21 [symbol] (h) | |
| | | | | | | | | (human) | | |
| 12 | PIGR | P < 05 | P < 05 | | 1282.66 | 19 | AIPNPGPFANER | T(mouse | 20.73 [symbol] (m) | |
| | MOUSE | | | | | | (603-614) | human) | T(h) | |
| 14 | P0N1 | P < 05 | P < 01 | P < .01 | 1853.84 | 82 | IFFYDAENPPGSEVLR | up | 2.23 [symbol] (m) | up |
| | MOUSE | | | | | | (209-305) | (mouse) | 0.77 [symbol] (h) | |
| 6 | RETBP | P < 01 | P < 01 | P < .01 | 1226.73 | 71 | YWGVASFLQR | | | |
| | MOUSE | | | | | | (108-117) | | | |
| | | | | | 1360.67 | 57 | QRQEELCLER | | 7.19 [symbol] (m) | |
| | | | | | | | (172-181) | | | |
| | | | | | 1789.83 | 63 | WIEHNGYCQSRPSR | up | | up |
| | | | | | | | (185-198) | (mouse) | | |
| | | | | | 2079.98 | 132 | LQNLDGTCADSYSFV | | | |
| | | | | | | | FSR (140-157) | | | |
| 16 | SAMP | P < 05 | P < 05 | P < .05 | / | / | / | up | 6.00 [symbol] (m) | up |
| | MOUSE | | | | | | | (mouse/ | 5.63 [symbol] (h) | |
| | | | | | | | | human) | | |
| 5 | SPA3K/M | P < 01 | P < 05 | P < .01 | 2270.23 | 28 | AVLDVAETGTEAAAATG | | | |
| | MOUSE | | | | | | VIGGIRK(361-384) | | | |
| | | | | | 2366.01 | 66 | ISFDPQDTFESEFYLDE | | | |
| | | | | | | | KR(218-236) | | | |
| 10.1 | TTHY | P < 05 | P < 01 | P > .05 | 2517.24 | 178 | TLGISPFHEFADVVFTA | | | |
| | MOUSE | | | | | | NDSGHR(101-123) | | | |
| | | | | | 869.45 | 41 | FVEGVYR (84-90) | | | |
| | | | | | 1382.63 | 91 | TSEGSWEPFASGK | T(m) | | |
| | | | | | | | (56-68) | | | |
| | | | | | 2438.26 | 193 | TAESGELHGLTTDEKF | | | |
| | | | | | | | VEGVYR(69-90) | | | |
| | | | | | 1382.63 | 54 | TSEGSWEPFASGK | up | | up |
| | | | | | | | (56-68) | (mouse) | | |
| | | | | | 2597.36 | 43 | HYTIAALLSPYSYSTTA | | 8.92 [symbol] (m) | |
| | | | | | | | VVSNPQN(124-147) | | 0.94 [symbol] (h) | |
| 10 | TTHY | P < .05 | P < .05 | P < .05 | 2438.26 | 135 | TAESGELHGLTTDEKFV | | | |
| | MOUSE | | | | | | EGVYVR(69-90) | | | |

[symbol]

indicates data missing or illegible when filed

1. A Protein regulated by increased c-myc activity in the liver for use as a biomarker in the diagnosis, prognosis and/or monitoring the treatment of liver cell dysplasia (premalignant stage) or hepatocellular carcinoma (HCC), wherein the protein is selected from a first group consisting of Polymeric immunoglobulin receptor, Phosphatidylinositol-glycan-specific phospholipase D, Alpha-fetoprotein, Antithrombin 3, Apolipoprotein E, Apolipoprotein M, Fibrinogen beta-chain, Haptoglobin, Paraoxonase 1, Retinol binding protein, Serum amyloid P-component, and Transthyretin, or from a second group consisting of Afamin, Glutathione peroxidase 3, Hemopexin, Major urinary protein, and Serine protease inhibitor A3K.

2. The Protein according to claim 1 for use as biomarker in the diagnosis, prognosis and/or treatment monitoring of dysplasia or cancer, colon, lung and stomach dysplasia or cancer wherein the protein is selected from a first group consisting of Polymeric immunoglobulin receptor, Phosphatidylinositol-glycan-specific phospholipase D, or from a second group consisting of Afamin.

3. The Protein according to claim 1, wherein the marker is a body fluid marker selected from the group consisting of blood serum or plasma or a tissue marker selected from the group consisting of a immunohistochemical marker.

4. An Antibody specific for a protein according to claim 1 for use in the diagnosis, prognosis and/or treatment monitoring of liver cell dysplasia or HCC.

5. An Antibody specific for a protein according to claim 2 for use in the diagnosis, prognosis and/or treatment monitoring of dysplasia breast cancer, colon cancer, lung cancer, stomach dysplasia, stomach cancer, leukemia, glioblastoma or neuroblastoma.

6. An Antibody according to claim 4 for serum profiling in the diagnosis, prognosis and/or treatment monitoring of liver cell dysplasia, HCC, dysplasia or breast cancer, colon cancer, lung cancer, stomach dysplasia, stomach cancer, leukemia, glioblastoma or neuroblastoma.

7. An SiRNA reducing the expression of a protein selected from the first group
according to claim 1 for use in the treatment of liver cell dysplasia or HCC.

8. A Nucleotide sequence coding for a protein selected from the second group
according to claim 1 for use in the treatment of liver cell dysplasia or HCC.

9. A Method of detecting liver cell dysplasia or HCC, or of predicting the susceptibility or resistance to liver cell dysplasia or HCC, comprising testing a sample isolated from the liver or from body fluid, of a subject for the presence or concentration of a protein according to claim 1.

10. A Method according to claim 9, wherein the sample is tested for the increase of a protein selected from the first group according to claim 1, the decrease of a protein selected from the second group according to claim 1, or both.

11. A Method of detecting the response to a compound in the treatment of liver cell dysplasia or HCC, or of predicting the responsiveness to a compound in the treatment of liver cell dysplasia or HCC, comprising determining the presence or concentration of a protein according to claim 1 in a sample isolated from the liver or from body fluid of a subject treated with a compound selected from the group consisting of c-myc activity modulator, siRNA according to claim 7, or nucleotide sequence according to claim 8.

12. The Method according to claim 11 , wherein the sample is tested for the decrease of a protein selected from the first group according to claim 1, the increase of a protein selected from the second group according to claim 1, or both.

13. A Method to screen for and to identify a compound for modulating c-myc activity in the liver of a subject suffering from or being susceptible to liver cell dysplasia or HCC, comprising the use of a protein biomarker according to claim 1, an antibody according to claim 4, or both.

14. A Method of qualifying the c-myc activity in a subject, comprising determining in a sample of the liver or in a body fluid sample of a subject suffering from or being susceptible to liver cell dysplasia or HCC at least one protein selected from the first group according to claim 1, least one biomarker selected from the second group according to claim 1, or both, wherein the level of the at least one protein of said first group being significantly higher, the level of the at least one protein of said second group being significantly lower, or both, than the level of said protein(s) in the liver or body fluid of subjects without cancer associated with increased activity of c-myc is indicative of increased c-myc activity in the subject, and optionally further comprising the method of claim 13 to screen for and to identify a compound for modulating the increased c-myc activity in the liver of the subject.

15. A Method, for predicting the response of a liver cell dysplasia or HCC patient to the administration of a c-myc activity modulator, wherein the level of at least one protein selected from the first group according to claim 1 being significantly higher, the level of the at least one protein selected from the second group according to claim 1 being significantly lower, or both, than the level of said protein(s) in the liver of subjects without liver cell dysplasia or HCC associated with increased activity of c-myc is indicative that the patient will respond therapeutically to a method of treating cancer comprising administering a c-myc activity modulator.

16. A Method according to claim 11, wherein the c-myc activity modulator or compound for modulating c-myc activity is selected from the group consisting of anti-c-myc siRNA, inhibitor of c-myc/max dimerization, Sorafenib, Sunitinib, Gefitinib, Erlotinib, anti-HER1 antibody, anti-HER2 antibody, anti-HER3 antibody, anti-HER4 antibody, Trastuzumab (Herceptin), Cetuximab, Panitumumab, Matuzumab, Nimotuzumab, MDX-447, and Pertuzumab.

17. A Method according to claim 9, comprising the steps of
adding lysis buffer to a serum or liver tissue sample isolated from a subject suffering from or being susceptible to liver cell dysplasia or HCC;
separating the proteins of the lysed serum sample by 2D gel electrophoresis;
excising from the gel at least one 2-D spot containing a differentially regulated protein;
adding digesting buffer to the at least one excised sample; and
determining the identity of the protein by analyzing the digested 2-D spot by mass spectrometry.

18. A Method according to claim 9, wherein an immunohistochemical analysis, a western blot or both is performed for determining the presence or concentration of the protein according to claim 1, and wherein an antibody according to claim 4 is used, and wherein dysplastic or malignantly transformed cells isolated from liver tissue by laser microdissection are used.

19. A Method as claimed in claim 9, wherein peptide mass fingerprinting is performed, for determining the presence or concentration of the selected protein.

20. A Method according to claim 9, wherein the expression of the gene coding for the selected protein is determined by means of a PCR.

21. A Kit for the use in qualifying the c-myc activity in a subject suffering from or being susceptible to cancer or dysplasia comprising at least one standard indicative of the level of a protein according to claim 1 in the liver, or of the level of a protein according to claim 3 in a serum sample, of normal individuals or in the liver or serum of individuals having liver cell dysplasia or HCC associated with increased c-myc activity and optionally at least one antibody according to claim 1, and optionally at least one primer pair for determining the mRNA coding for the protein, and instructions for the use of the kit.

22-23. (canceled)

24. A composition for diagnosing or treatment monitoring of dysplasia or cancer, associated with increased c-myc activity in a patient, comprising an effective amount of at least one biomarker selected from the group according to claim 22, or comprising at least one antibody directed against said at least one biomarker associated with increased c-myc activity in a patient.

25-27. (canceled)

28. A method of qualifying the c-myc activity in a patient suffering or being susceptible to cancer or for classifying a patient suffering from or being susceptible to HCC, comprising determining in a body fluid sample of a subject suffering from or being susceptible to cancer at least one biomarker selected from the first group according to claim 1 and/or and optionally at least one biomarker selected from the second group according to claim 1, wherein the body fluid level of the at least one biomarker of said first group being significantly higher, the body fluid level of the at least one biomarker of said second group being significantly lower, or both, than the level of said biomarker(s) in the body fluid of subjects without cancer associated with increased activity of c-myc is indicative of induced c-myc activity in the subject.

29. A Method as claimed in claim 28 for predicting the response of a cancer patient to a method of treating cancer comprising administering a c-myc activity modulator, wherein the body fluid level of the at least one biomarker of said first group being significantly higher, the body fluid level of the at least one biomarker of said second group being significantly lower, or both, than the level of said biomarker(s) in the body fluid of subjects without cancer associated with increased activity of c-myc is indicative that the subject will respond therapeutically to a method of treating cancer comprising administering a c-myc activity modulator.

30. A Method as claimed in claim 28 for monitoring the therapeutic response of a cancer patient to a method of treating cancer comprising administering an c-myc activity modulator, wherein the body fluid level of the at least one biomarker of said first group before and after the treatment, the body fluid level of the at least one biomarker of said second group before and after the treatment is determined, or both, and a significant decrease of said body fluid level(s) of the at least one biomarker of said first group, a significant increase of said body fluid level(s) of the at least one biomarker of said second group after the treatment, or both, is indicative that the subject therapeutically responds to the administration of the c-myc activity modulator.

31. A method to screen for and to identify drugs against cancer associated with an increased c-myc activity comprising determining in a body fluid sample of a transgenic cancer mouse being treated with a compound to be at least one biomarker selected from the first group according to at least one biomarker selected from the second group according to claim 1, or both, wherein the body fluid level of the at least one biomarker of said first group being significantly lower the body fluid level of the at least one biomarker of said second group being significantly higher, or both, than the level of said biomarker(s) in the body fluid of an untreated transgenic cancer mouse is indicative of the therapeutic effect of said compound as a c-myc activity modulator.
